SEQUENCE LISTING (49 sequences)

SEQ ID NO: 1 - DNA, 39 bp, Homo sapiens
agcagaatgtcctgaaggcagctgcccccagtctacttg

SEQ ID NO: 2 - DNA, 38 bp, Homo sapiens
tgcccgaattcgccatgcgggaggcagccttcatgtac

SEQ ID NO: 3 - DNA, 36 bp, Homo sapiens
taggtctcgagtcagcctttgcccccaccaatgctg

SEQ ID NO: 4 - DNA, 20 bp, Homo sapiens
gacggtgaagctggggaaac

SEQ ID NO: 5 - DNA, 37 bp, Homo sapiens
caatgaggtaatccttggggttcgcaggtgccaaagg

SEQ ID NO: 6 - DNA, 35 bp, Homo sapiens
cctggagctctcatggaatgtcagctacacccagg

SEQ ID NO: 7 - DNA, 19 bp, Homo sapiens
attttgcggtgagtctctc

SEQ ID NO: 8 - DNA, 19 bp, Homo sapiens
  misc_feature 10: n = A,T,C or G
attttgcggngagtctctc

SEQ ID NO: 9 - DNA, 19 bp, Homo sapiens
tctgcaggacgcctggagt

SEQ ID NO: 10 - DNA, 19 bp, Homo sapiens
  misc_feature 10: n = A,T,C or G
tctgcaggangcctggagt

SEQ ID NO: 11 - DNA, 19 bp, Homo sapiens
gcagatctcgtggtgctct

SEQ ID NO: 12 - DNA, 19 bp, Homo sapiens
gcagatctcatggtgctct

SEQ ID NO: 13 - DNA, 19 bp, Homo sapiens
attttgcggtgagtctctc

SEQ ID NO: 14 - DNA, 19 bp, Homo sapiens
  misc_feature 10: n = A,T,C or G
attttgcggngagtctctc

SEQ ID NO: 15 - DNA, 19 bp, Homo sapiens
ggacgaaacgactcctcag

SEQ ID NO: 16 - DNA, 19 bp, Homo sapiens
  misc_feature 10: n = A,T,C or G
ggacgaaacnactcctcag

SEQ ID NO: 17 - DNA, 24 bp, Homo sapiens
gacactgcctattttgcggcgagt

SEQ ID NO: 18 - DNA, 6 bp, Homo sapiens
gtttga

SEQ ID NO: 19 - DNA, 23 bp, Homo sapiens
agcaggacgcctggagtgaccag

SEQ ID NO: 20 - protein, 16 aa, Homo sapiens
  VARIANT 9, 10, 11, 12, 13, 14, 15: Xaa = Any Amino Acid
DTAYFAASXXXXXXXV

SEQ ID NO: 21 - DNA, 36 bp, Homo sapiens
gacactgcctattttgcggacgcctggagtgaccag

SEQ ID NO: 22 - protein, 12 aa, Homo sapiens
DTAYFADAWSDQ

SEQ ID NO: 23 - protein, 27 aa, Homo sapiens
  VARIANT 6, 7, 10, 14, 24: Xaa = Any Amino Acid
ENADLXXLWXDGDXAYFADAWSDXKGQ

SEQ ID NO: 24 - protein, 27 aa, Homo sapiens
  VARIANT 7, 14, 15: Xaa = Any Amino Acid
ENADLVXLWTDGDXXYFADAWSDQKGQ

SEQ ID NO: 25 - protein, 27 aa, Homo sapiens
  VARIANT 12, 14, 16, 18: Xaa = Any Amino Acid
ENADLVVLWTDXDXAXFXDAWSDQKGQ

SEQ ID NO: 26 - protein, 27 aa, Homo sapiens
ENADLVVLWTDGDTAYFADAWSDQKGQ

SEQ ID NO: 27 - protein, 11 aa, Homo sapiens
  VARIANT 2, 5: Xaa = Any Amino Acid
IXGRXDSSGIR

SEQ ID NO: 28 - protein, 11 aa, Homo sapiens
  VARIANT 2, 5: Xaa = Any Amino Acid
IXGRXDSSGIR

SEQ ID NO: 29 - protein, 11 aa, Homo sapiens
  VARIANT 2, 5: Xaa = Any Amino Acid
IXGRXDSSGIR

SEQ ID NO: 30 - protein, 11 aa, Homo sapiens
IEGRNDSSGIR

SEQ ID NO: 31 - DNA, 15 bp, Homo sapiens
tattttgcggtgagt

SEQ ID NO: 32 - DNA, 15 bp, Homo sapiens
actgagcaggtgcag

SEQ ID NO: 33 - DNA, 15 bp, Homo sapiens
ctgcaggacgcctgg

SEQ ID NO: 34 - DNA, 15 bp, Homo sapiens
ctgcaggacgcctgg

SEQ ID NO: 35 - protein, 603 aa, Homo sapiens
MREAAFMYSTAVAIFLVILVAALQGSAPRESPLPYHIPLDPEGSLELSWNVSYTQEAIHFQLLVRRLKAGVLFGMSDRGELENADLVVLWTDGDTAYFADAWSDQKGQIHLDPQQDYQLLQVQRTPEGLTLLFKRPFGTCDPKDYLIEDGTVHLVYGILEEPFRSLEAINGSGLQMGLQRVQLLKPNIPEPELPSDACTMEVQAPNIQIPSQETTYWCYIKELPKGFSRHHIIKYEPIVTKGNEALVHHMEVFQCAPEMDSVPHFSGPCDSKMKPDRLNYCRHVLAAWALGAKAFYYPEEAGLAFGGPGSSRYLRLEVHYHNPLVIEGRNDSSGIRLYYTAKLRRFNAGIMELGLVYTPVMAIPPRETAFILTGYCTDKCTQLALPPSGIHIFASQLHTHLTGRKVVTVLVRDGREWEIVNQDNHYSPHFQEIRMLKKVVSVHPGDVLITSCTYNTEDRELATVGGFGILEEMCVNYVHYYPQTQLELCKSAVDAGFLQKYFHLINRFNNEDVCTCPQASVSQQFTSVPWNSFNRDVLKALYSFAPISMHCNKSSAVRFQGEWNLQPLPKVISTLEEPTPQCPTSQGRSPAGPTVVSIGGGKG

SEQ ID NO: 36 - DNA, 1812 bp, Homo sapiens
atgcgggaggcagccttcatgtacagcacagcagtggccatcttcctggtcatcctggtggccgcactgcagggctcggctccccgtgagagccccctcccctatcacatccccctggacccggaggggtccctggagctctcatggaatgtcagctacacccaggaggccatccatttccagctcctggtgcggaggctcaaggctggcgtcctgtttgggatgtccgaccgtggcgagcttgagaacgcagatctcgtggtgctctggaccgatggggacactgcctattttgcggacgcctggagtgaccagaaggggcagatccacctggatccccagcaggactaccagctgctgcaggtgcagaggaccccagaaggcctgaccctgcttttcaagaggccctttggcacctgcgaccccaaggattacctcattgaggacggcactgtccacttggtctacgggatcctggaggagccgttccggtcactggaggccatcaacggctcgggcctgcagatggggctgcagagggtgcagctcctgaagcccaatatccccgaaccggagttgccctcagacgcgtgcaccatggaggtccaagctcccaatatccagatccccagccaggagaccacgtactggtgctacattaaggagcttccaaagggcttctctcggcaccacattatcaagtacgagcccatcgtcaccaagggcaatgaggcccttgtccaccacatggaagtcttccagtgcgcccccgagatggacagcgtcccccacttcagcgggccctgcgactccaagatgaaacccgaccgcctcaactactgccgccacgtgctggccgcctgggccctgggtgccaaggcattttactacccagaggaagccggccttgccttcgggggtccagggtcctccagatatctccgcctggaagttcactaccacaacccactggtgatagaaggacgaaacgactcctcaggcatccgcttgtactacacagccaagctgcggcgcttcaacgcggggatcatggagctgggactggtgtacacgccagtgatggccattccaccacgggagaccgccttcatcctcactggctactgcacggacaagtgcacccagctggcactgcctccctccgggatccacatcttcgcctctcagctccacacacacctgactgggagaaaggtggtcacagtgctggtccgggacggccgggagtgggagatcgtgaaccaggacaatcactacagccctcacttccaggagatccgcatgttgaagaaggtcgtgtcggtccatccgggagatgtgctcatcacctcctgcacgtacaacacggaagaccgggagctggccacagtggggggcttcgggatcctggaggagatgtgtgtcaactacgtgcactactacccccagacgcagctggagctctgcaagagcgctgtggacgccggcttcctgcagaagtacttccacctcatcaacaggttcaacaacgaggatgtctgcacctgccctcaggcgtccgtgtctcagcagttcacctctgttccctggaactccttcaaccgcgacgtactgaaggccctgtacagcttcgcgcccatctccatgcactgcaacaagtcctcagccgtccgcttccagggtgaatggaacctgcagcccctgcccaaggtcatctccacactggaagagcccaccccacagtgccccaccagccagggccgaagccctgctggccccaccgttgtcagcattggtgggggcaaaggctga

SEQ ID NO: 37 - DNA, 30781 bp, Homo sapiens
cgattttttttttttacagttctggaggtcagaagctctaaaatcaaggtgtctgcagagctggctccttttgggggctctgggaggggaatctgttcccaccttttccagctaccagaggcctccacatcccttggctcatggcccctgctccgtcttggaagcagcctcctctccttggaccccatcaatgcatctccttctctctctcgtaggacctttgtgatgacatggggtccacccagccaatccaagaccacctcccacctccgggtccttaatctaatcacactgaatgtcccttccccaggtagcctacccgacttgcagggatctggacgtggccattgggggacatccctctgtctgtcacaccacacgccagaagctcagagagattccagggagggaggcggattctcatgttctccgagaccctcctgggccagagccctgcctgttggaccctgtgatgtcagtgggtgcagcagcccccgagatggatgcgggtgagggcagcccggggctgtctcgctagccctcactgcatcggccccattctgggtcaatggtaggtcaacctggtggatctctgaggtgaccaagcttactgacagatgaactacagcctcaggaagagggtcatggctggcaggagccagcggggggcaagaccagcagccctgagtgctggtgcaggccacgcagggcccagcctctgtagccagaagtcagcgccagtgccggtcctgagcaagatggccatttcacagatgaggaaagaaggctcagggctgtggggactgtccgggtgggtcacatggccggcaggtttcagggcctctctttccatctggtgcccacagttacgctgtggcttgggtgtggtctggagctactgcctcaggacccaccaccatcctggcagtgtggtcccttcagaaaagctgaaaatgcaaaaatcaggcacatgcacctccccccatgagctgctcaagagagaggagcagtcacgcatccttatggagaaaaggagaagcaggacccagaaagggtttccctgtaagattcctcagggcccagcaggcctcagccccaagattgaggccaaattattggaggggtgtgtgtgtgtgtgtgtgtgtgtaaaactgagtatgtgagcatgagcccagagctgggggtgcaggccacgcaggtgtgagagtccacctccccatctcgctgacccctgtgtatggaggagagatggaggcgggggaggctgtgtcctgcccaggccagccgcctgccagtgacagtggaggacttgctcacagtggggctgcttggggtcacacaggagtggcctggcaggcctggcagccccagccctggagccagccacagactcctgggttctgcttccctgggagcttccaggggagtgtcactggggctcacgataccgaccccttctgggatcagtggccccttgcttttggcacagagcttcagggccagagggtgcctctaatccagtagctgaggaaagctttcagtggtgctctgggcacctctgtgacctgccctgggcccagtcctcatgctgatcccactgtagccaccctactcagtgccccaggccctggacacctcaggccatgggatatcccaagcattcaaactctcacggccacctacgcttatgagcagaggcagaggcatggtgccaccgctgcctcctcaggcaggatatgctttttgtttgtttgttttgagacagggttgttcgctgtcattcaggctggagtgcagtggtgtagtcagggctcattgcagcctcaaactcctgggctcaagcgatcctcccgcctcagcctcccgagtagctgggactacagtcttgtgccactatgcctggctaatttgtttttatttttattttggtagagactgggtcatgctatgttccccaggctggtctcgaactcctgggttcaagcaatcctcccgtctttgcctcctaaagtcctgggattatagaggtatgagtcactgtgcccagcccagaatgtgcctcttaactttcataccgcagcccctatgctcaggacgaggagggacataggccagctgccccaagttccccaaggggactgtgccaccctggagtgggctctgtgatggacagagtcaccgtctcccagcagctcccaatccatcccatcacaagtcaggtctaggcactgccatcagactgatagttgaatttttcctgccaagaggaggtccctaagagcctgagccacaggcgagggacagtgctgtcctgtggccaggccccgttgtcactggggagtggcctggcctgttcactcacccagctccagtctgcaccaccgtcgctccacttggggttgtcactgcctgaggttaggacgtcagcagaagccagcctcatgtggcagaggcgatggggaattacagcctgcaaaggctgatggcatcagagagattcaggaaagcaaagggcttattaaaaagctgaaattggttctaaacaaggtcagccgtcgatgcgccttggagggaagccaggcgctgagcagcaggaatggggcttatttcctgacaccgtggccgtgacaccctgcacctccccggcctaccccacccccaaccccactcccacccccattcccatccccacccccaccgccacctctggagggcaacgtgcacccagacactggagcagaggcagcaggggtgggagcagggaggaaagccccccgcggggcaccacagggaggcatgtggccaagagagcacctggcaagcaggtgtcacctctgaggagggtgagggcaggaggagagctgactgttgcttccttaccctggggtcggattgctcccttttaaaatccattaatgcaaggaagggcgcagccacggggccatccgattggattcgctccctcccgcccggccccagtctccgcagattttctctgcggttgcatttttcatctgagcgtcccttcctcttgactccagtggaatttttctgaccttggctgacaaggtgtgcagccccctccagctcagggcaccctctttggggtgcttgccccaggcactggcccaccctgtgagctcaggcgcattgtctcgcctctccgaggctgactctcctgtgtctgagacagggatgatcgcacacccagccacccaaggctggccaggatggaggcagggcaggggaagccctggtgcggcctgacttgtgtctcctgtccctccctaggacttagttatcagtattttggaatcagctgaatcatttctgtcaaatgctagaggaaagccagttttgagttggataaagaaaatgtctttgattcctgttaaaaataaacatgtgtgcataacactgctgagcacgagaccgcacctgccgtccctccttagagctcccagcagctgttggtctttggcgtatccgggagaggagcgaggcctggaggtgtgttgagccgtccaagggctggtagcatcaggggctcttcactgcctctcgctccacagaaccgctgggttgaaggaagctggagggatcaattgtccttgtcatccaacttcctgtgtctgtagatggggaggaggcgctgggttggaaagtgcttctctgaggacgccagctgcgcattcaggagggcggagcaggatatgaccccagagctctgaccccggaggagcaggaggggtgcctggggcctcaataccccagaggggagagaggtgccccccttcccacaacgcgggccccagctggtgctcgtttagcctgttgtctagctggagccacctgggtggccaccaggctgctgggagggctcacccctgagctgggtgtgctgtggagcccgtgaccctcacctctgcctctgctggcttttgttatcccgtgctgggaacaccagtgcctgcccaccccggaggctcttcgtatcaccggctcgctgtggccgctccccagtcccgagagtgaatgtccgggatgcagcagacacctgcttacaaggcacaggtgaggaggttttggaactaatctgaggcatgaaggtgaggtgctctcaggcgacggaggtggcatgtcctgatgggaaagccaggccttcggaggctgagacccttcccaaggctcaccagagaactcccaagagtgtctttcccctggcctgggagacaatttcacaccaatgccacactctattgcacaggtgattatgcacttggtcctcacctgcctgggctgtgggtgggggcgaacggtgcccctctgggtgcaatcagtggggaggggctcatggcctgagcttggctttgcaggatggcctggccactgtggggcagagagcactgcctgcgagggccaggggaaggctggcactttctccaaatttcacgttcgtgcaaagacacagtcattcctttctacagcgtagagctcagagctgaagcagcctccagacctgctgtcatgggtatttaaggacctaaatgttgctgtcagcctatgacatgaatgtgcccctaaggctagattctgggtttctccagagagacgagaaacaggagggaaaaggaaggaagggagggaggaggctggggaggagggacagcttctagtccagctggagagatctgtcaacccagcctggggggtggagctggagggatcaagcagaatgtcctgaaggcagctgccctcagtctacttgcgggagaggacaggagggagaggtgccgtggtgagactgaccctcgggcccacgggtggatgatggcaaaggtcctgtggcaggtgtgggagaagcagggctgcggccagctgccctgagaggcttcaaattcaggccagatctgctctgggcaagaggaagctgtgggtttgggagcttcagagacaggggagcaggcctgtcactcaccctcttttccttaaaggctgtcacccccagcagtgcaccctgcagcctgcctatcccctgtgcagctccagctccgtctgtccccagcaatgcaccccacgcccatgtcccccactccctatgatgctctcgcgccttctggagcagcagtggtaccagctggaggctgtggaaaaattgagaggagacaaaagtgggaagagcaggccgtggagagagtgtctaaacacagggactatttggagattgtcttatttttggtttgaaatgggcccacctgctagtattgtacaaacggctctgctcgctaggccaggcctgcagtggctacgggcctatccttcactggtcagcctggagcagctcctcggacctcagtctgctcatccgtgggtagaggcgacgcagaccccatcccactggggactgggccagacagcctgtgaggcagtcagctggtgcctggccagagggtgtctgaaaggcctcttggctgcagggtgcatctgcttttgggacagctcttcagagccatctcagaagggacagcatccgcctgtctacttcaactcccactgatgacgtccatgtgtcattagtgccaattagaggagggcagcaggctgagtgcttggcctggggcgcaagcttgtgggagggaaaattggattccccgctagacaaatgtgattacccgtgctgcctggacccaccccattcaggaccagggcataaatggccaggtgggaccagagagctcaccccagccatgcccgccctcagtcgctgggccagcctgcccggccccagcatgcgggaggcagccttcatgtacagcacagcagtggccatcttcctggtcatcctggtggccgcactgcagggctcggctccccgtgagagccccctcccctatcacatccccctggacccggaggggtccctggagctctcatggaatgtcagctacacccaggaggccatccatttccagctcctggtgcggaggctcaaggctggcgtcctgtttgggatgtccgaccgtggcgagcttgagaacgcagatctcgtggtgctctggaccgctggggacactgcctattttgcggtgagtctctcctccctgccagctctccaaacccttcctgacccggcaccccatctggccgtctttctgcactcaccctccttaacccagaaagttcttctgtcacctctcagtgtttgagttgactctgctctgagccaagtctgcaccccgagcttgggcatcatggggatctcagtttgaggacaaaataggaaagacatgctttccatggtccttgactgcccgccccaaatcatgggacccagtaatcccaaggaatggaactatacacagaggacgcacacaccacacacactcgtgccgctccacaccccacacgcgcatgagcacagaccccacgaccctcggccctgtaaacaatgcctagtaccacgtccccagagcacacatgcccactgatgtgggccaggtggtggcgcgtggctgggcacagcatgaggcctgcacacccatgggatccagcctgacctactgtgcttgtacccgtggaccgactgagcaggtgcagacaccctgcccctacccataggtgttgaacaagccccacagggctcagccaaaactgttaggatgggctcttgactcctttccgtccaaatttacctccactcttgtttcacagggtggagaaaggggaaaagtttcccttcaaactggaggggactttaggcccttccacaagctgcagggggtccccgaagtgggcgagcccaccgcctacaatgggtcctgctccagccttcctttccacatgggactggccaccagggcttctttgtggatgaaacccaactctctccacagaaacacacaaaggagtgacaaaaaagttttatttctgaagtcaggaggtgcctgctttatctgggagtgccgagctggagcgggggagctgtgtctgtgcggagcggccggctccagctctgcatcttggtctcaccatgggctggtggggcggagagtccctctctgcctttttgggtgttgtgaattcatgagggcagatggtggctgctgtttctggagctgagcttgccccctccaattcccaagactcggccaggaggcacttgcctgtgtcgctagtgaggcttagggccacccaggtagggtgagtggagctgcctgggaagggccctgctctgacccatagggctgcacagattcctgtgcacaaagacactctgtggctcaggttccagccaagaaagacctgggacagccctgattctgagccgtcagccgtctggaggagcagctgaaccccatcgctgacagtttggggttcttcgcaagcctctgccatgaggcaccctgagtgcaggagcatttgctcctctctggggcagcgtcccccctgcctgcatttcccggctgctagggaagccagaggaagccaggtgactttgcctttccgtgcactggcaaggttaatttgcacaacaaattcctacgaaagcaaatgtcagccttattattcctccctaagcaatatgttcaactaatatcttcttgatttccagctgaaatatcacaggcgaactttctgcctctgtttaaaatgagtctctcggcaaaccagcggtgtccccagcggggttgcattgtgtggcttttttctctagggaaagttttgcagtatcttttgcagatttcttttttctcaaggaaatgaagaaaagatgttttaccctgaatgtaagattaaaaagaaacacaacccaaagtccttaaggccaagggcccgaggctttcttggggtttccatgggaaaggccaggcagggcagggaagcagcttcgtttggacaattctggtgggctctgggtggctggggtggtctctagttgcctggttcctggccctagggtgatttagggttggggcaatattggcctggggtgtgagagtcaggtggaggaggtagctagggatatgggcttggtgtgggtcggaatgttaggaatatgattctcacacacgcgtgaatgttggagggcaggacaggtgtaaacaactctgaccatctgtttgagccttcaatcaattaatagatgccaaatagacaaatatggaatctaagaagggcccttcttgagtacgaagggcagcctgcttgttggggaaggctcctcgctgtctctcagtgggtctccctggtggtcttccatgtgacctggcctcgggccaccgtgtccctgccccttagggcccagtgcaaggggagcagccggagatggggagggcagaggtcactgatccagatcccaggaagcatcctcggagaatgcagcaggtccaatgcctccctgggcctcagtttaccttctccaagcagggctgggactaaggtgaggggaacaatgcctaggctgcagggtttaaagtggtgcttcaggctgggtgtggtggctcatgcctgtaattccagcactttgggagggcaaggcaggaggaccgcttgaggccaggagttcaggaccagcctaggcaacatagcgagaccctatctcaaaaaaaaaaaaaaaaatccaaaattacatgtatttataaagtaataaagcagtgcttgttctcagggttgtgccagtgcagggtgggcacctgctatcaatcaccaccttagatgtggcaccctggtttctctctcctttcctgttcatgtcccaccttgttcccaactctcccctttctgaggctccgcaactctcctgcctgcatgaatgtgggcagcattggtctaaggcattgttcccagtcctgaggaggccttgggagcctcaggcatcttcttaagaagctcagtgtcccgcccaccaggcccgggaatgcccatggctaacagaacctgtgacctgctggcaagtgacagccctggttcctggctgcagaggaggaaccaactccactgtcggggctcatctccactgtctccaggcagaaatggagatagaaggtgtgagtgaagccacagccccaggcagaaccctcagatccacagggactgaggcccagcatccccagatcagctggcaatgaatgcggagctctgccggggaatgccctcttcccctgtggattggcccggcttggctccttcatgcctggagcccagtgcttgtctctgcaggacgcctggagtgaccagaaggggcagatccacctggatccccagcaggactaccagctgctgcaggtgcagaggaccccagaaggcctgaccctgcttttcaagaggccctttggcacctgcgaccccaaggattacctcattgaggtaaggggtggccgcgagtacccaggagggcgtgggctgcgtgtatcatgctgccatcctgtgccaatgtcatagtacctttcctgtccctgataagtctggggcctgggcctggccagctatgacagagagaaagccaaggaggatggccagaggcagtggtggggccaaagcacttaggatggtccagctctgctattgcccctgagccctcaaagacgcagctctttgccaacacgtagctgacatggtttctagatgcggggcttgtgctgaagcctctcccattggactagaccttgcttctggatgtctctctcctcctggtcctgttaaccatcaggtgggattctggagcccaggcgacctggctttccatcccggccctgttgccagctcgctgtgaggctcgggacaactgcccccctcctctggacctcactttgcccatctgtaaaatggagatgggaactagatgatctgtatggcctgaccaactctgggagttcatgggagccataaaacattgcaaaagccataggcttcttatgctgctctaagagccccccaacctcttagggaaagcaacccaccttgcccttgaagacaaaactcttttaactctagcacacctgacagcttttgtcttgtgtaagtaatcctttggtgtggcagtggcctgggctggccgttagtgccaggtcttaatcatgggtgtagctgagtgaccacacgggaggccagtgccatcgaaagattgattgcatttccggagagaaggggcacctgtgccgtgcagggccacctgcagagccctgggtttggtcaggaggcagatgcaggagctaggggagagcccaggcctgaggctttcttggggtttccatgggaaaggccaggcagggcagggaagttgcttcatttggacaattctggtgggctctgggtggccggggtggtctctagttgcctggttcctggccctagggtgatttagggctgaggcaatattggcctggtgtgtgacagtcaggtggaggaggtggctaaggatatgggcttgggacgggttggagggttgggaatatgattctcacacaagcgtgaatgttggagggaagggcaggtgtaaacaactctggccatctgtttgagcctgcaatccattaatagatgccaaatagacaaatacggaatctaagaaaacagcacagctggctgtttgccagatgaggaaactaaggcccggagacggggagtggatgtacccaggggttcggggtgtgctggggcagacctggtgccagaagaggtgttcttccaggaggttgttgagcccttaccctgcttcccagtggacgaggcaagggtgagcagagaaccctctggaagcacctcacagggggaggccaatgcctgtgtgtgctcagcccagtggccccaggccaggtataggggcctttgcctggagttctgggcgtcccctgcacagggcggcccatcggctttccgcaaactcaaagagcaaattagccacccacacgggtctgcactgtggagtgcccagaaagctatgaaaggcactttccacacccgatccccgtttccattcatgacagcatgtgaggaagccagccccacagcttgcctcctgagtctgcctgtggcctccagcaccgcgtagatccatggagctttctgtggctgcttggtcccctcctgccaagctggtggtgactacataatatagcgtggctccgcagctcagcgtgaccatcagcaagccccagggtgtttccttcccttcatagagggcactgaatcctcaggctgggctgctgaactgaccaatgatcccccacacccagtccccagtgaccttggggttggtggagggaacaaatgttccggccacactggctcacggcgggcccagtagtcactggttgaatggatgttaagtgaatgtaggtggaatgtgtttagaagagaaaagtatttgatggccggtgcaaataggggatcctgggggtcgtcagcccgggatttggccccagccagggccatgcaagcctcaagggtcccttattcacagagatgagagtgaggtgtgtcacctggtaggtgtgggtgggcagggatgtggcatcctctcaggagcccaactctgggggctctgagagggcgaccagctgaaccctgtctcggctgcaggacggcactgtccacttggtctacgggatcctggaggagccgttccggtcactggaggccatcaacggctcgggcctgcagatggggctgcagagggtgcagctcctgaagcccaatatccccgaaccggagttgccctcagacgcgtgcaccatggaggtccaagctcccaatatccagatccccagccaggagaccacgtactggtgctacattaaggagcttccaaagggcttctctcggcaccacattatcaaggtacgtgcgggtccagggccgaggtcctcgcccagccctgccttcctcccgggcctgggttgtccctgaccctggagagctgtccacagtcctggttggaccaggtgtcctcttatcactggaacctcaggcacctgcctgagcaggggagcagcgagtggtcttggcttccccatcactctgctcactcccctaccctcaaagggccttggagctcccacttccctgcctggaccaaagtgaccaacatcttgacttctgcatctttccttgggactggaactgctgtctgtctgccaggccacctcccccttcatgaaaaagcagctcgagtcagaactcacttgctctcgaacacccgtccggggaagacagaatagcaaaggcgatagctgtcgagtgtccactatgccactagtcccgggggaggggcgaggtgatccctccttcatcataagagcccctagtttccagcagaggaagtgtggcccagggaggttgagtgtccagggtcacatagcaagaaggggcatgtccttgaaggtgcactcagagaggttatgtgagtgtccagagtcacacagcaggaagaggcatggccttgaaggtgcgctttgggcccgggctctaacctcagggctgccctgcctcccactggggctgcaggagctggcccggccacagccccatatgcatgaggacggctgcgtcctgtccctgccttggcaggaaatggcctgtacgaacctcatttccttcactctggagctgcctacgggatttctttctgggctgatggctccaccctcaaggctgtgaaccccagaagtgcccctgaaattgtctggatcatccctcccattttacagatgggcattcggaagcccatggaggagggctgctggggaggggagggtgggcggccggttcccgggctcagagggctgcctcctcacagtacgagcccatcgtcaccaagggcaatgaggcccttgtccaccacatggaagtcttccagtgcgcccccgagatggacagcgtcccccacttcagcgggccctgcgactccaagatgaaacccgaccgcctcaactactgccgccacgtgctggccgcctgggccctgggtgccaaggtgcgtgccctgcgaccccagcatggtgtctcctgcctgggcccctggcatccccacacctctgtttccccagcttcaccgtctcagaggcttcaagaaggggctcccaagggggctcacgaggccaccagaagggccaggcctgaggtggccccctcgcctctgtgatgtctgaaatgcttcaagccttttttttattttccacagaaacgcaagtgccccaggaccttatgccctcagtgggtcctgattctgtttcccaactggagtcagggttttgtgtacaggtggtcccagggctggctggcggtggggccagtgttgaggcctccacaggctgagacgatgggggtctccccagctcttacacctgtctagagaaggggttttgggggaaggctcagccctaggcaccagctcctctccccaactccctgacccgagtctcacaggatgttcctggggcgtgtaatgagctgcctgtcaggaggaagcatcgctctaatcctgctgcgccccctccaccacccctgaggctcaggccccaacagttgactgggtttgcccctgcccagacctggggccctctcaggacacacctgtctgtctgacaccttgccccacacaggcattttactacccagaggaagccggccttgccttcgggggtccagggtcctccagatatctccgcctggaagttcactaccacaacccactggtgatagaaggtaggcggctctgctgccatcctcctcagaagccctaggactcagctgtgttgagccagtgagcaaatcccacccttcgtctgcccagcatggtgcccccgctgtacagctcctcttggcacgaggcccttgcgtctgcctcatccgctgtccatcttccatggctgtggtgggctcccagggacaggaccccgaggggctcactcctcacagcttcgccagggcccagcagtggccccacacctccctcctcgtccctataatacgggctgccatcggcgcagcactgtgcatcccaggggaaaggacaaggagaattttcataagggaagctgccagatgccaagttccaaatacagggagaggtgtgtgcataaacagggaggtggtagtaaagcagggcatattccaggacacaggcgctccggcctggccggagtgcagggacgggaaggggagctggggcagctgctgttggatcacccatacctgagggtggagtgagcagggtcagcttttagcatggcaaggccaggtgatgctgtctcagaaactgaaaaaaatacttccctctctctgttgcaccagcatctttcaccatatatagacacacacaaacacaatgcacacacacacgcacacatgcacacacatgtacacatatgcacgcagaagcacacacatgcacacactttttctctctctctgaatggtgcttcttcttttcatttgtgcaaggtgcagaagtggcctaagagataggtgctaggctgtgcctcaccaccttggggacgctgggtggatgacctcacccgctgcctgttctcccacctcccacagctattgtgaagctgggaaaggctcacgcacagaacttgctgtgggcacggggctgtgctcagctaacagtcgcctgtggttttttttttttcctggctgcataagtaataccttccgccactgttggagttatctccacatgcacagagaagggtataagtcagcttgggccttagcatgacacgggatttattgctcatttaaaccctgaaagcaaatggatttttgaaaactcctgaagcatcagggaagtctgtctcttcatgattatctggttgtatttatggatcctttgccaaatataaggacgctcctgtcacttcacccatcttgagatgggctgcttttctataaatatcaaaatcccacaattatttcatgatttaatccccatctgaattttaaccaattgcaacaccctttttattattatcctctgatgtaatcagcctggagatacaggagccgtttgagtttggatggtgtaggggtgtcgagggaagaaggcagaggcaagaggagttgttttggcctcatcttaaaggaaacaagtagggagatggttgggccccctggaagtgccctgcaccccagtccacagccctggctttgaacctcagcttggccacttcccagccctgtggtcttgtgtggagtcttcagttcaggctccatcctcagtttcttaatctgtgaaatagggctgatgattctcactgtgcaaaacttccatgaaaggggtaggttatgcttagagcatttttggcacccagggaagatgttagtatctgtaccaccctcttggtttgatcagaaagggcaatgcatgtacactggtgagaagatggacagcagaaaagcatgaagaagatgaaaccaccccccagctcccggctccaagttttgaagtgagcactggtcacccagctatgtgtctgtcctgactgcttttctggcccattaatttttttcttgtgactagaattttatatctttttttttcattgttgtttgtttttttacttaaacttcatcagaagcctcttccctgtgaattaaaaccccttgtcaatttcacactccttgccgcctgacatcgctttcctgtacataacccagggggcagagcctcctgctgcacagcccctccagaccaccccgccatgtccaccatgtccaccatgcatccgggggcatggggccaggcctttagatcctccgccaaccatgagcccagccggtactcagagcctggctgctgattcactaggcatggagtttcaggaacggtggggctacctggatgacacctggttgtgaggatgttcgagtgggtgccagcatccctgaacctctgtttttttgttttgagacgaaatctcactctgtcacccaggctggagtgcaatgcacgatctcagcttaatgcaacctccgcctcccaggttcaagcgattctcctgcctcagcctcctgagtagctgggattataggtgcgtgccaccacacctggctaatttttgtatttttagtagagacagggtttcgccatgttggtcaggctggtctcgaactcccaacctcaggtgatccacacacctcggcctcccaaagtgctgggattacaaggcatgagccaccgtgcccggccccctggacctctgaccatcagtttcatgtgaccactgggaggctggcccagggagcttcagggactctccaattagcaaacggcaaggagaactaacaggtgtggccacaccataatcaggagagacagtctccgtgactcgaagaagcaccatgttattcctttggaagaggcccttgagtgtgggctgaggctcataaagggacagcaagtcatctgagagcacacagcacgctgttgaccaagcaggacctggcctgtgcatgaacgggggctagacctgcaacgctggccctcctcctggggggccacattctcactcaaattgctcctgagatgtcagcttggtaggtgggttccttgggtctgcaggacataaatcaaagtagtaaaagtgggggctgctgggaggatgtgctccttgaatgcttcttgttaacctcaacacagagatacactcacacttgcccagctttgacgcagggccagggctcttctgtgccatcggcagatgggagttccatgccctcttctttgcccagaccacaccctgcaggttgtcaccgtcctgcaggctgatttccagcttaggttcctttgaacgtggaagaagaacatcaaagcacttatgaaacaatctgagcacagaactcaagttgtttacgagtttccgtctatcagcagcatctcacccgcacctgattccatggaggctttaagtggctcacgttgatcacatctttccatagcattcaactagttttcatagaaacagcagctctttcttcccagtgagtgtagcctgtttgcgttaattgatcttgcgagctgcctggcatccacaggtgtggaaacaaagacagccagctcttggtctgcctagcaggtgggagcagatggggggtgaccggccctgcctcctggctgagggtggctggggtcatagggataatctgtccgcagggggaagtgagagggcctccacttaccgctcacctccatccatcccaccttctcccaggacgaaacgactcctcaggcatccgcttgtactacacagccaagctgcggcgcttcaacgcggggatcatggagctgggactggtgtacacgccagtgatggccattccaccacgggagaccgccttcatcctcactggctactgcacggacaagtgcacccagctggtgagtggggctgggcccggcactgcaccctccctcctcccgcgtccctcagtggaggcctggcaggtcgtggcccacgaagggtggcaggcacagctttggtttcccctgaccctgaatccccctgcggtcctccctcttttctgtatgcaagggaaccctgctgctgagaggccatttgtgtgtgtatgcacctccctctccagcaataaccatggctcccactagtccacacccacgtgccaggcttccacgagctgcattttaaaatatgccttcattatgtaagcaacacacacatgcacttgccactcacaagcgcaggcactgcagctaaagagaacgctcccctcatccgctgccgcctccccctgagatagccacttcttggctctttgtgtccttccagacctttttcctgtatatttcaggaagctcttgcaggtgttagaaaccttttgtgtttttaaaaagttgtattgaatagttaacaaatttttcaacaaatgggaggcatttttctggtgatttgcctttttattgctgcatagtattccacaggatggggttttcaccacgcacctgagcagggcaggactaggtgaggcagtgaagtgccgggtacaggatctcagggagcacccgtcctcagggctatgcacgctgggtggaggcacaggttggcgcttagactggcgccaacagcaggtcctgtccttggcttctggtgagttcagagagtgactactaaaatctcctcctttgcttgccctatcttggtccaagcctgtgctgaactgtctccctatcaatcagtgtctaggctgttctcaacttttcaccaatagaaagaagcttctgggaaatccttgtccatgtaggaaaccattttctcatgtagatttctaggcatgaaattgctgctcagagatgtgagtgttcccttgagtaaatcttgcaaaatgcccctcagaaagcccatcttctgctccctcccacacataggacgagagggtgcccggttttactgacccactggattattaatattgtaaatattttctgtcatttgggaggaagacatgtcagtgttgtttaacttgaattttctatttgtggacttgaggatcttttcctctgcctcttggccatttgttcctgttttctgttcattcctatgctcggtttttccagtaggtggtttgtttttcttactgttggtttctaggagttcttcttatatgatggatgttatttcatagtttgtgtatatgttacaaacatttcctcctgtgcggtagctttacttctcacagtgacctgcaaagtaagtgttgacgtacccatttcacagatggggagactgaggcttgctgggatcacactgccagcagtcacgcagagtgaagattcaaataccagtctgtctgactttgaaagctgtggccgtgacagcagatttcgttgtaatgaggtttagagtggattttccttcctagtaaatgggtcgattgggatttgttcttcatgtgggtgagcccccagagcatcccagtcttccaagcaaaggttcctcaaggctgaagggagacggagctctcagccggcatggcacccattgtctccggcaggaaatcagctccttttgtcccagacgggggtggacactctctcccacttccctaaagcgcccactggtgaagcgtgggcccatgtaaaaggcaccttggccagggctcatggggaagagcggcttccggccaggcagcactcactgccccttgcagggccttggggccttcgaccccgctgaccgctgactgtgagaacgcctctctccagcctcaggaatacagacccgcctccccttgccagagtcctccactaccttaagtaacagaatgagaatgttcagtcttgtctctcctttggcagataaaagaggggcacaaattctgcagccaaaatctggcctagtctagctatcccagtgaaataggccaagacaagagtgctgctttggaccagagtgcccagaagagcatagtcctccctccacctggccgacacctggcatctctgggccagggtcggcctctgggggccaaaggggcattgggaccctttgggctcccaggcctggcacacagtggcctctcaagccattgcaagggacacaaagctataggcgacggcaaggtcagggcggccgctgcttgggagcgagagggaaggagcaaaagtgagtcctaaaaagctcggccatctgggaccttcagggatggggctgtcgggaccttgagctctgggacctgctgactgcatgaacaaactgggcagagcctggcagcgggtgggcaaaaggacaactgactcacaaagaaaatgctcgatttcaggaaagacgtgttcttttctttccttctttccttcttttctttcctttcttttctttttctttctttttctttttctttcttttctttcctttctttctttccttccttccttccttctcttttctttcttccttcctttctttttttttttttttgatggagtttcgctcctgttgcccaggctgcagtgcaatggcacaatcttggctcactgcaacctctgcctcccgggttcaagtgattctcctgtctcagcctcctgagtagctgggattacaggcacgtgccaccatgcccggccaatttttgtatttttagtagaggcggggttttaccatgttggccagactattctcaaactccttacctcaaatgatccatgtgcctcagcctcccaaagtgctgggattacaagcatgagccaccatgcccagccatggaaatgcattttctatctaaaaaaagttagcattaggttagagaaaactttcgttctctggaagattctctcatgccccagtgatggcataagacctcttgagatgagggctgccatcttttctgtgaggccggcctgcagcctcagagtgtccagctgcagcaccccatggtcacatccatcttggtcctggatgaccaccttccagttgtagctacagctcccagctgtagcacggggtgatgctgagcacagggagaatgcagccttggctgctttgaagctgaaaggaagccacaacgcagggcagctgaaggtcctgctcggcccaccacgtaggggacctgccaagtaccaccttctgccacttgcttttccctggacagcaagctcttcagcacagaggccacgcgctgctcagcttggtggctttgccacaccctgtccacgggaggctcgccttaaaggcctgttgactgagtgaagatgaacctgtcaggcttcaggggctcaggagaggatgaaggactcggaaagaaagtcgctcctgagtgggactaggggctgcttctttgggagctgggggctgttgcggccccctcgctcttccccgtgaggtttctgatggtggctctaacctggccggggagaaagacctagaaaatgcaagtgttccagggaagcaaactgcccagggtggctgctccctaccgggtcctggccatggacgggagggtcccctcgggggtcaggccctgacactgcagccccccgaccccacaggcactgcctccctccgggatccacatcttcgcctctcagctccacacacacctgactgggagaaaggtggtcacagtgctggtccgggacggccgggagtgggagatcgtgaaccaggacaatcactacagccctcacttccaggtaggaacctgcaccccacccctgccccgcccccacaccctgccaccacacgacctcctgggtctactgtttcctgacaccatagggatggctccaggctggcttctttttcctgggccagccccacccgccccccacccatgtggcctgtgctccccacttgggtgtctggtgtctgatcgtagggaggcctctggcacatggcagatggtggtgggattcgggagacccaggcggccctctgggaacatcaatcttggtcttggataaccaccttctggtttgaatctaatgccctccctggccagaagccacacttagcgggctctggcttccactgtagaggctggggcaaaatggacacccccagaggtcagggaggcctgagggaggacacagtggccgggggtctggtctgcagctctctgaggtctcctctcccccacccctcggctctgcctgccccaggagatccgcatgttgaagaaggtcgtgtcggtccatccggtgagtgcccagcgggaaggctgtcccactcactgccaccagctggggtggctgagagggctgggggtgccaccagaaggagagggacacagaaagtgatagggggagggagagccatccagggcagggggagggtgcctgtgggtggctctgggctgggctacctcatggggagactcactcaggagttcatggagagacgttggaaagagtggagcagacaggacactcccaggggacgcgtgtcctcagtctgatgggccgaccacaaacccgagagtgtttgggggagatgagggcactaaggcaggcagtggagccctgatcccacagagggccaagcccaggcctcggtggggacttgcccaagtcctttgagatgtgtcatggcccaccagctgcacccccactggcctgggccctggccctgttgtgacccactgggtcacaggagctgatggtttataacttgctcgggaacactcagcctgtgaccttcgcacatgaatctgctgggctccttgtagtggacgacagggactgtaccccagaggtgccgtggcatgcacagtggcgtggtcctatgggggcgaggcctccagcacctgccaacgccaggtggcaggtgctgatggtcacattggcttttcctcagggagatgtgctcatcacctcctgcacgtacaacacagaagaccgggagctggccacagtggtaagtcacccccgcttccccctgcacctgcccagggcgagtgttcagcctgagccatctgaggaaggatgacaggtcttgactcctcacttagggcatgggctcgtcccctcaataagccagtcgttctcagctccctgaaccacagtggcagcatcaccagggagcttgggatgaatgcaacctcctgggtgccaccccaggcccactgagtcagagacacaggcggtggcatcgggcaatccatgttttaacaagctcccagcggatgttgacccctgctcaaggttgagaagcactcacaccaagaagcttctcagcacccggggctggtgtggggagaagctgtaggacaagaggttgacctttgccccgtggagctcaaggtgcaccagggagacaggcctggagcacctgctgctaacaacaagcaaaattacaaaacaaaactttaaaaaccaggttgaggaggcagaggtgcggggacctgggagggccttgtcagaaatgtggctgtcacatggagcccaggggagaggggaggaggtgaaggaaggctctgtgcaggggctgcgtgcgggaaggcccagaggaccaagaacattctcaggatggaaaggagaggaggagagggtggagacatgggtgggggtcaggcaggatgagcgtcagctgaggatgcattttggctggactgcagacagttcaaggagaatctgcactggagagatctattttcaaggctgggtatgcaggcagcaccaggacacgggcagccacgtgatgcatcacgcagcggggctgcttccgggagccaaaggggatgctgttcacaattacctgggtcccaggcacaagccagggctgtcctggcaaatgggccaccttgccagtggatccgtgccagctgcttcggtaaccatggtggtctccaataattatcctgtgaattgtgtggctcaagtggagcagacctgggttacagctaccctgacatccctcagcgccaggaactcaggtccatgtcacagccctattctttgtgttttaatccaatgtggattcacctgatgtttactgagcccagactgccagtggtttattttattttattttatattttatttttttttgagacggagtctcgctctgtcacccaggctgtgtgttctcagcttgctgcaacctccacctcccgagttcaaccgattctcctgcctcagcctcctgaatagctgggactacaggcacccaccaccacgcctggctaatttttgtatttttagtagagatggattttcaccatgttggccaggctggtctcaaactcctcatgtaatccctcatgctgggattacaggcatgagccatagcacccagccagtgtttgtattttcacaggtgagacataaaggctcagagagggcaggcacttgaccaaagtcacacagctgatagatggctgagctgggatttgacccagggtcttgtgcctcacagctgttaccccacccttcctgtctcccttggatttcaggtgacaagtgtcctaggttcctgatcacaaactcagggtggagacaggacagctttgtcagtcctttattctctctagaggaggtgctcagtatatcacagcagtggctgatgtcgggtgcttgccacagctcagcactgctctaataactcatcctaaccacaccaagccttgctgagacctctgagcaatcccaccaggagatacagtgaggtccagccactcaggccacacagcgaacaggaggcagggctggtatgggtgcaggtgccaaccccgactgtgccgcctctcggctaagcacctgtgcagtgactgagtgggctcgcaaacagagggctctgcaggtggagcaccccaggcaagtccccaccatcagccccattctgcaggcgcggccccggggcaggggatgggagggtcttgccgggcatccagcgaggagactgcatattccctgcccaatctggcttcacggcggcggaactgacgccacgtcccagggcgttcggggctgccaggcctggtgagcaccgcggacatctcagatggccgttctgcaggttgaggccctcggtgccagcgtccccgtgggcagttctgtggcttccctggcccatgtcgtcctcttggcagtggccacatcacagcaccaggtgggatcaaaggggagggcagctccccaggtggacctgagcttctcacactctgacaagccctcgcctgctgatccaagacacagtggcctttgaaattcgatttccttccccaggtgaaagaggagaacagattctacacagggtcggactcagttcccttttgatgtctgtggtggcagagggtggggagggacaggaactcacacaggagggcaatcgtcccgatctgacgggggcgggatggatgagaaaagctgggaaaggaggaatccggcccagagaacaatgctggcaaaaacgcgtcctggccaacaggtgccaggtttcgtctctgggcagaactgtcccatgctctttgtctgcacgggaggagggggcggcagctggtgtaagtgccacgccaagctttgcccagtccctttgttctgccatagagtaaagattgcctttcttccccaccagccagatgaaatccccaggcccacagcagcttcacaaacagcggggagcctggaggggcaagggtggccagggcgtcatgtcccggcccctgacctgctgggatctgagctgctgccgggggagctttgctctctccagccccagctcctccaagctgctgctgttcttcctcctatggctgaggctccacggtcaccagctccaagagcagcaacccagcgaggggcttaaagctggcagtggggactcagagggtgcctgccctgcccctcccatgcgatggcaggccgtagggttgccgtgagaggctggaggttgtcgcggcctctagtcacggtcctgtcttatagagtccctgctcccctcagagccacgttggagaaatgactggtggacggtgcggggctgcagtggaaagggcctcccttgcagtccttggctccaggctctcgctggaggtcatggggacagcttccagacaatggccgggacgctgggtgcactcaggctgtgccaccgatgctgtggccaccacggggcatccccttgcctgctctgagactcagtttctgtacccataaaatgggcacaataacaccccatccgcttgtcccgtttcatttttcctcacagcatcatcagtacctgatacgaatttcctggttgactcactcactcactgcctgtctctcctgccaatggtggcaattccttgagggcagggactcggttccccagagcatgcctggcacggtgcagtgaacagacgagtggacgcagtgtacacgtgggtgcggcgaaagctgctggatggcagcgtctgcgtggcatggcccggggctgacgggtctcctccaacttgcaggggggcttcgggatcctggaggagatgtgtgtcaactacgtgcactactacccccagacgcagctggagctctgcaagagcgctgtggacgccggcttcctgcagaagtacttccacctcatcaacaggtgagggctccctgcacaagctccctgcccccagggaaccccgacacagaacctcgggcctgttaggcggctgggcagattggaggagtccaggctaagttctaggagcagagacctgtggcggcatcactcacccctcccctcactcgtttcctgctgagtctggatttggcttcagagcctcctcaacccagcctgcagcagccattacccacccgccaaggtgacaggagaggtgaggccgttgccggtgaaggcagcatctggggtggcctggcggaggcagcggggctggggaggaggtggcaggacggttgccccagggacaggactcgagttgcagggaggtgttgctggtgcccactgggctccctgcccgtcagctgctccccagcctggctgttccttgtccccaccaggttcaacaacgaggatgtctgcacctgccctcaggcgtccgtgtctcagcagttcacctctgttccctggaactccttcaaccgcgacgtactgaaggccctgtacagcttcgcgcccatctccatgcactgcaacaagtcctcagccgtccgcttccaggtgcgctgccatgggcccgggtggggcatgcagtcaggcaggcctcatggggggccccagtgaggggtgatgggtctgcactccaaactgctggcaaaagcactcgcacaagacaatgagagcaagtgccaggtggtgacacataggcctagacagccagccagccagcagagagagaggagagaggagagagaggagagagaggagagagaggagagagggagagggagagagggagagggagagaggagagagagaggagagagagagagagagagagagggagagagagagaaccaataacgaggcaaggaagggagggcaggcaccctctctggtgacacctccacactgtaccgaatgccaaatgcaggtggtgtgagcagggcgcactaacctgcctaaaaataaagcaccaggggaggggaaggtaagaaacgaaccagtctgaagctgcgagctggttttttcctccttatctgagacccactggtattcgaaggcatctaatttatcctgggcccactgggctgtggtcaggaggccagggcctatgcagagttagtcgcgtaaggtgccgcagcctggagatccaggaagatccttcccagaagcatttgggagccagattaactgctaagtcaaacatcctcagcaccaagggaaggaagggctaatactggcttgctgagaatagtgtggggtggacgtctgatggggttgaaaacttggcatttggtgtagccttgaggggaagagatagctaaaaaatatcagagcctgcagccaggggctctggttgctacactagggtgaatgattaaattgggtggggacagaggcggggagaggcctggatgactcccaggttcctgactaggtgaatgggaagccaggggagggggcatttactgaggtgaggacccccaaaaagcaacttgggggagcagagtgagttcagtttgagacaagctgagtttgagggcaagaatccaagaggtggctgggaagtggctggggaagcagccacccatcttgcccctccagcctcagtttacctcctgcccccttccttgcagggtgaatggaacctgcagcccctgcccaaggtcatctccacactggaagagcccaccccacagtgccccaccagccagggccgaagccctgctggccccaccgttgtcagcattggtgggggcaaaggctgaggggggacctactcctccccctcctccatgctgtccctgtgggctcacaccggcactgtgcactctactctgcgacgatccccatggaacagccctgcacgcccaggatgaaggggccagaccacgcccctgcctgagaccacggtccaatccagccttcttcccccagggtcccctgcatggctgagagggtgtgggtgccctgttgacctaccctggaccgagtggaccacgacctcgtccatttaaacccggctgactcagtgcagggacagcctgcacagtggtccagggtccagccctccgccagccctgttccgcctcactgggtgtggcctggcttctgggacaggcaccatgctgggccggggtgtggaatcaccgggaacgcccccgcccccgccccgctgctcccggtgtgcagcgggtgcgggtgccgcttaaacatttccctgctgagtggctcgtgtttcacagtgggcggcttccctgcgacggaggcaggaccaggcatttagctagttagagactcgcctgggaaattgctccattcctgagtaaacagatattttcgcccacctaaagggaagccctgacaacaactatcaccaaaagacgaggcggcaaagatccagcggggcttctgggcgccggttccacgtggggtggaattattagcaccagcttgcttctctgccggtggggccagcgctgaacagaccggggtggagtcagggctgtgctttccgcgtggttctgccacttagggagtgtgccttgggcgggccatttcacattcctgaccctcacttttctcatctgtaaaaccaggctgatgccgtgcgggctaatgagccaataaagctcacacttgggctggcacccactggaggtgggtatgtttgcactccgggccgtcgctccaggagagagaagcttgctcctgagcctcctccctgtgccaggcgtggtaccaagtgcttccccctgagcgtcctgaagctgcctcagctggcctgggtgggtctccccttcctgccctctgagctgcctctcagggacaagttcagctctttgatggtttctggagacagtaacagatgcacttgtgttcagccacttcagcaaaacgctgcccagcctcatggggagagagggagatggatgggcctcggagtcccctcgcatcagctaagcggctgctgcttctgcaaggcacttcacttcatcacagaagggaagtcacgagcccaggagctctgcctcacgttgtataaatcacagcttcttgtggtccataagtcacggcgctagcgctgtgaaatgccaggccccagcctggagagccactccggccctctgtcatctttacagcctaggtgacagtagacacggagcaggcccaagaagcctcttcccgcgatcacacccaattgccagcccagagaagggtcccagggtcctgcccaaacacctggggggcacttgtagcctgccgatctcgggcagggaaactgagactcccagataagtacctcacctggggcccaagagcggcagtgattgggaatcctagccagctctggaacctgccacagcgacaaccccacccccacctcaccccccactcacactcttgctctcaggctggttatcctcttcctcttgcagagacagcagccgtgtcccccctaccccgctccagcagtacccgcacctcctacacccagataagcgtaaccccgtggttgctgagagttgccacttgccccacactgaggctatgcacagccgtggcaatctcgtgaagccatgtagtttctgtttccattgatggaggaggaaactgaggctcagagacctaaagttaggtgccaaggtcacccagagtaagccggggagctcgaagtgtccccaggtctgactccagtgtctgaatgctcaacctctgcttacttaggaaacaggaatttccccaggacccttgtgggggattttatctaaaataagttccttatttagaggcttaggggaagtgctttgttacctgaaaatgacctacatgccccaagccaaagggactctgggctcgcccaccttccgaatgattggggtgtgagtccagcagcccaggttgctattcctgcccctgctgtgtgaccctgagttagttactttccttccctaggcttgggtctccctatctgcccaccgaggagagggttccccagcctcccagggggcagggactgctagggtggcacaaccccccatctgtgaggtgctcagagccctggggtgaggaatgctcatgcccatcagtgttgagagaagtggggcgaccccaggctccagccagcataggggtgcctgcccgactgggtcccagagcacaccagggctgtgctttctggtggctgctagcctcatgtggctacttacgattaattttaaattaattaaaatgatacaaggtcgggtgctgtggctcatgcctgtaatcccagcactttgggaggctgagatgcgaggatcacttgagcccaggagttcaagaccagcctgggcaacatagtgaaatcctgtctctacaaaaaatacaataattagggctgggcgcggtggctcacgcctgtaatcccagcattttgggagactgaggcgggcggatcacctgaggtcggtagttcgagaccagcctggccaatatggtgaaaccccgtctctactaaaaatacaaaaattagccaggctgtggtggtgcgcgtttgtaatcctagctatttgggaggctgaggtgggagaattgcttgacctgggaggtggaggttgtagtgagctgagatcacaccactgtactccagcttgggcaacagagtaaaaccctgtctcaaaaaaaaaacaacaacaacaaaaaaaaacaaaatggaacctttctgcgtcccattcatacccccaacttttcacatg

SEQ ID NO: 38 - protein, 11 aa, Homo sapiens
  VARIANT 6: Xaa at position 6 can be Valine, Methionine, or aconservative substitution for either Valine or Methionine or can be absent.
ENADLXVLWTD

SEQ ID NO: 39 - protein, 21 aa, Homo sapiens
  VARIANT 11: Xaa at position 11 can be Valine, Methionine or can be a conservative substitution for either Valine or Methionine,    or can be absent.
DRGELENADLXVLWTDGDTAY

SEQ ID NO: 40 - protein, 31 aa, Homo sapiens
  VARIANT 16: Xaa at position 16 can be Valine, Methionine or can be a conservative substitution for either Valine or Methionine or can be absent.
LFGMSDRGELENADLXVLWTDGDTAYFADAW

SEQ ID NO: 41 - protein, 41 aa, Homo sapiens
  VARIANT 21: Xaa at position 21 can be Valine, Methionine or can be a conservative substitution for either Valine or Methionine or can be absent.
LKAGVLFGMSDRGELENADLXVLWTDGDTAYFADAWSDQKG

SEQ ID NO: 42 - protein, 12 aa, Homo sapiens
  VARIANT 7: Xaa at position 7 can be Aspartic Acid, Glutamic Acid, or can be a conservative substitution for either Aspartic Acid or Glutamic Acid or can be Absent.
DTAYFAXAWSDQ

SEQ ID NO: 43 - protein, 21 aa, Homo sapiens
  VARIANT 11: Xaa at position 11 can be Aspartic Acid, Glutamic Acid or a conservative substitution for either Aspartic Acid or Glutamic Acid or can be absent.
WTDGDTAYFAXAWSDQKGQIH

SEQ ID NO: 44 - protein, 31 aa, Homo sapiens
  VARIANT 16: Xaa at position 16 can be Aspartic Acid, Glutamic Acid or can be a conservative substitution for either Aspartic Acid or Glutamic Acid or can be absent.
DLVVLWTDGDTAYFAXAWSDQKGQIHLDPQQ

SEQ ID NO: 45 - protein, 41 aa, Homo sapiens
  VARIANT 21: Xaa at position 21 can be Aspartic Acid, Glutamic Acid or can be a conservative substitution for either Aspartic Acid or Glutamic Acid or can be absent.
ELENADLVVLWTDGDTAYFAXAWSDQKGQIHLDPQQDYQLL

SEQ ID NO: 46 - protein, 11 aa, Homo sapiens
  VARIANT 6: Xaa at position 6 can be Aspartic Acid, Glutamic Acid or can be absent.
IEGRNXSSGIR

SEQ ID NO: 47 - protein, 21 aa, Homo sapiens
  VARIANT 11: Xaa at position 11 can be Aspartic Acid, Glutamic Acid, or can be a conservative substitution for either Aspartic Acid or Glutamic Acid or can be absent.
HNPLVIEGRNXSSGIRLYYTA

SEQ ID NO: 48 - protein, 31 aa, Homo sapiens
  VARIANT 16: Xaa at position 16 can be Aspartic acid, Glutamic Acid or can be a conservative substitution for either Aspartic Acid or Glutamic Acid or can be absent.
LEVHYHNPLVIEGRNXSSGIRLYYTAKLRRF

SEQ ID NO: 49 - protein, 41 aa, Homo sapiens
  VARIANT 21: Xaa at position 21 can be Aspartic Acid, Glutamic Acid or can be a conservative substitution for either Aspartic Acid or Glutamic Acid or can be absent.
SRYLRLEVHYHNPLVIEGRNXSSGIRLYYTAKLRRFNAGIM

(19) United States
(12) Patent Application Publication
(10) Pub. No.: US 2003/0040015 A1
(43) Pub. Date: Feb. 27, 2003

(54) Methods and reagents for identifying compounds and mutations that modulate dopamine beta-hydroxylase activity

(76) Inventors: Kwang-Soo Kim, Lexington, MA (US); Chun-Hyung Kim, Waltham, MA (US); David Robertson, Nashville, TN (US)

Correspondence Address: CLARK & ELBING LLP, 101 FEDERAL STREET, BOSTON, MA, 02110, US

(21) Appl. No.: 10/092,908

(22) Filed: Mar. 7, 2002

Related U.S. Application Data
(60) Provisional application No. 60/274,095, filed on Mar. 7, 2001.

(51) Int. Cl. G01N 33/53; C12Q 1/26

(52) U.S. Cl. 435/7.1; 435/25

(57) ABSTRACT

Disclosed are methods for determining whether a compound is an inhibitor of dopamine β-hydroxylase. Also disclosed are methods for detecting a change in a dopamine β-hydroxylase nucleic acid sequence.

### CROSS-REFERENCE TO RELATED APPLICATIONS

This application claims benefit from provisional application 60/274,095 filed Mar. 7, 2001, herein incorporated by reference.

### STATEMENT AS TO FEDERALLY SPONSORED RESEARCH

This work was supported in part by the National Institutes of Health (MH48866, HL56693, NS33460, (P50)NS39793, and RR00095) and NASA (NAS9 194983). The government may have certain rights to this invention.

### BACKGROUND OF THE INVENTION

This invention relates to the fields of drug discovery and disease diagnosis.

Norepinephrine (NE) is a key neurotransmitter in both the central and peripheral nervous systems. It is the principal sympathetic neurotransmitter, and an important modulator of diverse neuronal functions in the central nervous system regulating mood, attention, drug addiction, arousal, and cardiovascular function. Approximately ten percent of all prescriptions written in the United States are for drugs targeting NE or its receptors; these include antihypertensive agents, antiarrhythmic drugs, and antidepressants. NE is synthesized by dopamine β-hydroxylase (DBH) that catalyzes oxidative hydroxylation of dopamine to NE. DBH is unique among the catecholamine-synthesizing enzymes by virtue of its almost exclusive localization in the chromaffin granules of the adrenal medulla and the large dense-core synaptic vesicles of noradrenergic neurons. Vesicular DBH occurs in both a soluble and a membrane-bound form and soluble DBH is released into the synaptic cleft during vesicular exocytosis. Such synaptic release is presumably a major source of the enzyme present in blood and cerebrospinal fluid (CSF). Levels of DBH protein in the plasma and CSF are under strong genetic control. Linkage and association studies have established the DBH locus as the major gene controlling DBH levels in body fluids.

Given the fundamental role of NE in central and peripheral nervous system function, it was of great surprise and interest when adult patients lacking NE were described. Biochemically, these patients display characteristic perturbations in NE metabolism: undetectable levels of NE and its metabolites and approximately ten-fold elevation of plasma dopamine levels, suggestive of a metabolic block in the final step of NE synthesis. Consistent with this, DBH protein is undetectable in these patients by enzyme assay of plasma, by radioimmunoassay of DBH protein in the cerebrospinal fluid, or by immunocytochemical examination of sympathetic fibers. These NE deficient patients exhibit profound deficits in autonomic and cardiovascular function, but apparently only subtle signs, if any, of CNS dysfunction.

The important role that NE plays in cardiovascular and sympathetic nervous system function makes NE an attractive therapeutic target for the treatment of disorders characterized by sympathetic nervous system dysfunction. Recently, pre-clinical and clinical studies have demonstrated that chronic sympathetic activation in congestive heart failure (CHF) is a maladaptive response that accelerates the progressive worsening of the disease. Consequently, therapeutic interventions that inhibit sympathetic nerve function are likely to favorably alter the natural course of the disease.

CHF develops in approximately 465,000 people per year in the US. In men and women with CHF, the 5-year survival rates are 25% and 38%, respectively, according to the Framingham study. In the US, CHF is the most common discharge diagnosis for Medicare patients. It is estimated that approximately $10 billion in health care costs are spent annually in the US for the treatment of CHF. These figures indicate the staggering economic impact of this disease on society. Recent clinical studies with carvedilol, a β-adrenoceptor blocker, reduced mortality and the risk of death and hospitalization in CHF patients. Unfortunately, the therapeutic value of β-blockers is limited by their propensity to cause acute hemodynamic deterioration. This deterioration is caused by β-blockers abrupt inhibition of sympathetic nerve function. Thus, although the introduction of β-blockers represents an important advance in the treatment of CHF, a less abrupt means of modulating the sympathetic nervous system would be highly desirable.

Inhibitors of NE biosynthesis represent a promising alternative to β-blockers. DBH inhibitors can directly modulate sympathetic nerve function by inhibiting the biosynthesis of NE via inhibition of DBH. DBH inhibitors provide several important therapeutic advantages over currently available therapies; first, DBH inhibitors could be expected to produce gradual modulation of sympathetic nerve function, in contrast to the abrupt blockade induced by β-blockers, thus eliminating the need for dose-titration; second, low doses of DBH inhibitors could preferentially inhibit NE release in the heart since the storage pool of NE in this organ is selectively depleted in CHF; finally, inhibition of DBH could augment levels of dopamine. This could have a beneficial effect on renal function in CHF. Previously described DBH inhibitors include disulfuram, FLA-63, SCH-10595, fusaric acid, BRL 8242, SK&F 102698, and Nepicastat (RS-25560-197). The clinical development and therapeutic utility of some of these compounds has been hampered by their low potency, lack of selectivity for DBH, and toxic side effects. Thus it would be desirable to develop more potent and selective DBH inhibitors.

### SUMMARY OF THE INVENTION

Here we report the identification of seven novel variants including four potentially pathogenic mutations in the human DBH gene (SEQ ID NO:37) from analysis of two unrelated patients and their families. Both patients are compound heterozygotes for variants affecting expression of DBH protein. We have shown that in these patients NE deficiency is an autosomal recessive disorder that likely resulted from heterogeneous molecular lesions in DBH.

In a first aspect, the invention features a method for determining whether a compound is a potentially useful DBH inhibitor by contacting the compound with a region of a DBH polypeptide that includes an amino acid that corresponds to amino acid position 87, 100, or 331 of human DBH (SEQ ID NO:35) and determining whether the compound binds to the DBH polypeptide region.

In a second aspect, the invention features a method for determining whether a compound is a potentially useful DBH inhibitor by contacting a compound with a region of a DBH polypeptide that includes an amino acid that corresponds to amino acid position 87, 100, or 331 of human DBH (SEQ ID NO:35) and detecting DBH biological activity (e.g., NE biosynthesis).

In a preferred embodiment of the second aspect of the invention, the method for determining whether a candidate compound inhibits DBH biological activity involves detecting NE biosynthesis.

In other preferred embodiments of both aspects, the DBH polypeptide contacted with the candidate compound is of mammalian, most preferably human, origin. DBH polypeptides are preferably at least 90% identical to SEQ ID NO:35, more preferably 95%, 97%, 98%, or 99% identical to SEQ ID NO:35. Exemplary polypeptides that can be employed in the contacting step of either the first or second aspect are those that include one of the following sequences: SEQ ID Nos: 38, 39, 40, 41, 42, 43, 44, 45, 46, 47, 48, or 49.

Compounds identified using methods of the instant inventions are potentially useful for the treatment of a patient with congestive heart failure, or chronic activation of sympathetic nerve function.

Further, compounds of the invention that inhibit DBH biological activity can increase dopamine levels, and thus can improve renal function in a patient with congestive heart failure.

The invention also features isolated polypeptide regions that include the sequence of one of the SEQ ID NOs: 38, 42, or 46.

The invention also features a method for determining the nucleic acid sequence of a DBH polynucleotide obtained from a patient, by detecting a change in a polynucleotide sequence at the consensus donor site located between the first exon and first intron, or in a polynucleotide that encodes a region of the polypeptide of SEQ ID NO: 35 that consists of either amino acid position 87, amino acid position 100, or amino acid position 331. A change in a DBH polynucleotide sequence derived from the patient identifies that patient as carrying a mutation in a DBH nucleic acid sequence. Such patients may be at increased risk of having a pregnancy that results in miscarriage, still birth, or fetal or neonatal death. Such patients may also be at increased risk of having noradrenergic disease, depression, dementia, bipolar disorder, schizophrenia, or attention deficit/hyperactivity disorder.

By “polypeptide” is meant any chain of amino acids, regardless of length or post-translational modification (for example, glycosylation or phosphorylation).

By an “isolated polypeptide” is meant a polypeptide that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source; by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “substantially identical” is meant a polypeptide or nucleic acid molecule exhibiting at least 90% identity to a reference amino acid sequence (for example, any one of the amino acid sequences described herein) or nucleic acid sequence (for example, any one of the nucleic acid sequences described herein). Preferably, such a sequence is at least 95%, more preferably 97%, and most preferably 98% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformed cell” is meant a cell into which (or into an ancestor of which) has been introduced, by means of recombinant DNA techniques, a polynucleotide molecule encoding (as used herein) a polypeptide of the invention.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide of the invention, or an RNA molecule).

By “binds” is meant a compound or antibody which recognizes and binds a polypeptide of the invention but which does not substantially recognize and bind other molecules, unrelated to the polypeptide of the invention, in a sample, for example, a biological sample, which naturally includes a polypeptide of the invention.

By “derived from” is meant isolated from or having the sequence of a naturally-occurring sequence (e.g., a cDNA, genomic DNA, synthetic, or combination thereof).

By “a region” is meant some part of a whole, for example, a region of a polypeptide that includes at least 40% of the length of the full length polypeptide, more preferably at least 50%, 60%, 70%, 80%, 90%, 92%, 95%, 97%, 98%, or 99% of the length of the full length polypeptide. The minimum number of consecutive amino acids that forms a region is at least 30 amino acids. Polypeptides that include amino acids 87-515 and contain the putative catalytic core domain (Oyarce, J. Biol. Chem. 276:33265) are also included in the definition of “a region.” Specifically excluded from this definition of a region is a full-length DBH polypeptide (e.g. human DBH, SEQ ID NO: 35).

By a “DBH polypeptide” is meant a polypeptide, or region thereof, having at least 90% amino acid sequence identity to SEQ ID NO: 35. This region is not required to have DBH biological activity.

By a “DBH nucleic acid” is meant a nucleic acid that encodes a DBH polypeptide.

The invention provides targets that are useful for the development of drugs that specifically inhibit DBH biological activity. A method for developing potent and specific inhibitors of DBH by targeting functionally important regions of the DBH polypeptide provides an improvement over existing therapies. DBH inhibitors can be expected to produce a gradual modulation in levels of NE, will preferentially inhibit NE release in the heart, and will be more selective for DBH than existing drugs. In addition, the methods of the invention provide a facile means to identify a mutation in a DBH encoding nucleic acid region. Such mutations may indicate that the patient has an increased risk for having a miscarriage, still birth, or fetal or neonatal death. In addition, such mutations may indicate that the patient is at increased risk of developing a noradrenergic disease, depression, dementia, bipolar disorder, schizophrenia, or attention deficit/hyperactivity disorder.

Other features and advantages of the invention will be apparent from the detailed description, and from the claims.

### BRIEF DESCRIPTION OF THE DRAWINGS

FIG. 1 shows the catecholamine biosynthetic pathway when DBH activity is deficient. The absence of DBH enzyme results in greatly elevated levels of dopamine, the precursor of NE, and its metabolite dihydroxyphenylacetic acid (DOPAC). NE and subsequent downstream products in the pathway are undetectable in plasma and CSF. DOPA is dihydroxyphenlalanine, and DHPG is dihydroxyphenylglycol.

FIG. 2A shows the putative disease-causing mutations in DBH deficient Patient 1.

FIG. 2B shows a haplotype diagram for Patient 1. Patient 2's parents are unaffected. Patient 1 is a compound heterozygote for mutations IVS1+2T→C and D100E inherited from his mother and father respectively. Open symbols denote affected, and solid symbols unaffected individuals. In the haplotype diagrams, letters denote nucleotide positions as indicated, with numeral “1” representing the wild type, and “2” the mutant allele. In the electropherograms, the upper and lower lines of text represent wild type, and the affected patient's sequence, respectively. Exons are depicted in upper case and introns in lower case type.

FIG. 2C shows the putative disease-causing mutations in DBH deficient patient 1.

FIG. 2D shows a haplotype diagram for Patient 2. Patient 2 is also a compound heterozygote, having inherited mutation IVS1+2T→C from her father, and mutations V87M and D331N (in cis) from her mother. Patient 2's parents are unaffected. Open symbols denote affected, and solid symbols unaffected individuals. In the haplotype diagrams, letters denote nucleotide positions as indicated, with numeral “1” representing the wild type, and “2” the mutant allele. In the electropherograms, the upper and lower lines of text represent wild type, and the affected patient's sequence, respectively. Exons are depicted in upper case and introns in lower case type.

FIG. 3A shows analyses of mRNA splicing products from the wild type and mutant constructs. The left hand side of the panel shows an RT-PCR analysis of DBH mRNA from COS-7 cells transfected with a construct containing either the mutant (lane 1) or wild-type (lane 2) alleles, using primers complementary to sequences in exons 1 and 2 (open arrows). The molecular weight marker is indicated in lane M. On the right hand side of the panel is a schematic representation of normal and aberrant splice products. The IVS1+2T→C mutation generated both normal and aberrant transcripts. Open boxes represent exons, dashed and solid lines indicate spliced and retained intronic sequence, respectively. Exonic sequence is in upper case, intronic sequence is in lower case, and amino acids are depicted below in bold face type. The solid arrowheads indicate the position of the IVS1+2T→C mutation. This analysis demonstrated that the aberrant transcript used a cryptic donor splice site, and retained 505 bp of intronic sequence, which contains a premature stop codon.

FIG. 3B shows a comparison of the nucleotide sequences of the splice donor and acceptor sites used in the normal and aberrant transcripts. Both transcripts from the mutant allele were isolated and sequenced. The cryptic donor splice site is indicated in bold. Nucleotide positions of the G residue of normal and cryptic splice site are numbered in relation to the A residue of the start codon. Nucleotide sequences spliced out in normal and mutant alleles are underlined.

FIG. 4 shows an amino acid sequence comparison of mouse (SEQ ID NOs:23 and 27), rat (SEQ ID NOs: 24 and 28), bovine (SEQ ID NOs: 25 and 29), and human (SEQ ID NOs: 26 and 30) DBH, D. melanogaster tyramine β-hydroxylase (TBH) and human peptidyl-glycine amidating mono-oxygenase (PAM). Human DBH sequence is indicated in bold. Only the identical amino acids are shown, whereas gaps are marked with dots and non-conserved residues are marked with dashed lines.

FIG. 5 is a table showing the plasma levels of catecholamines and their metabolites in controls, patients with autonomic disorders, and patients with DBH deficiency.

FIG. 6 is a table showing the sequence variants identified in DBH deficient patients.

FIG. 7 is a table showing the clinical features of six reported cases of DBH deficiency.

FIG. 8 shows the human DBH amino acid sequence (SEQ ID NO: 35).

FIG. 9 shows the human DBH cDNA sequence (SEQ ID NO: 36).

FIG. 10 shows the human DBH genomic sequence (SEQ ID NO: 37).

FIG. 11 is a table of regions that comprise an amino acid corresponding to amino acid position 87, 100, or 331 of SEQ ID NO: 35.

### DETAILED DESCRIPTION OF THE INVENTION

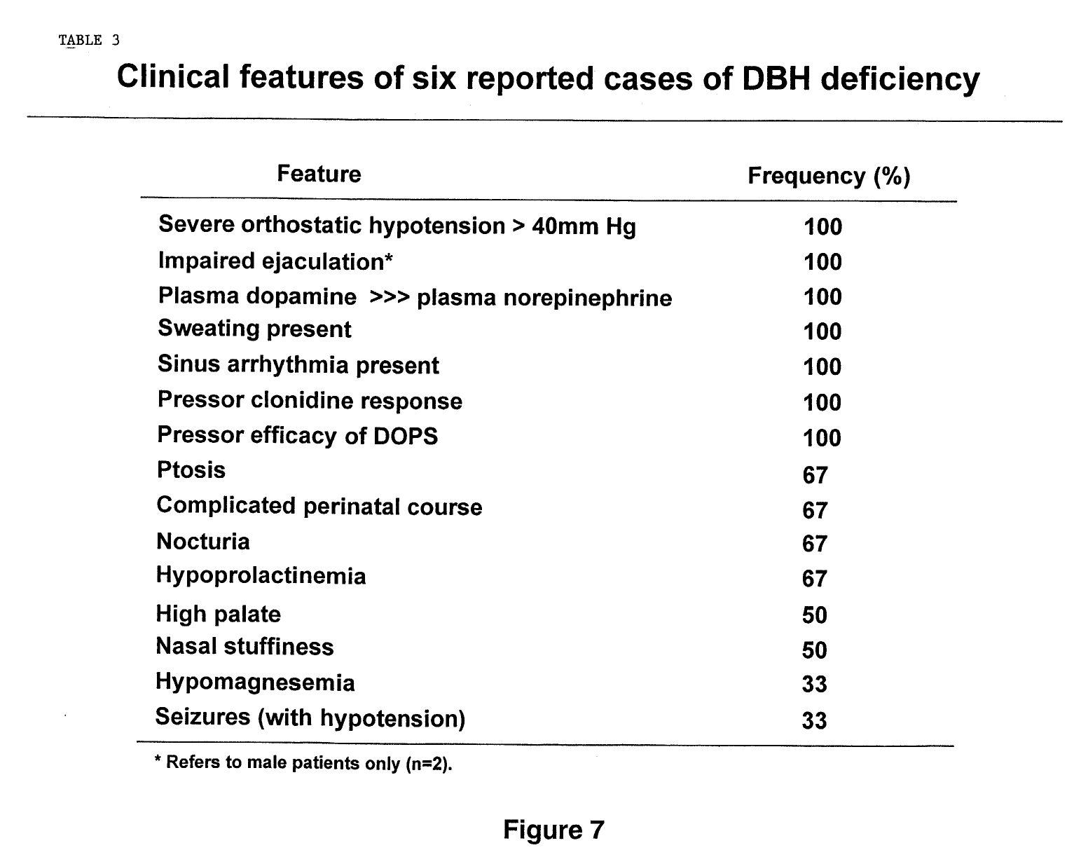

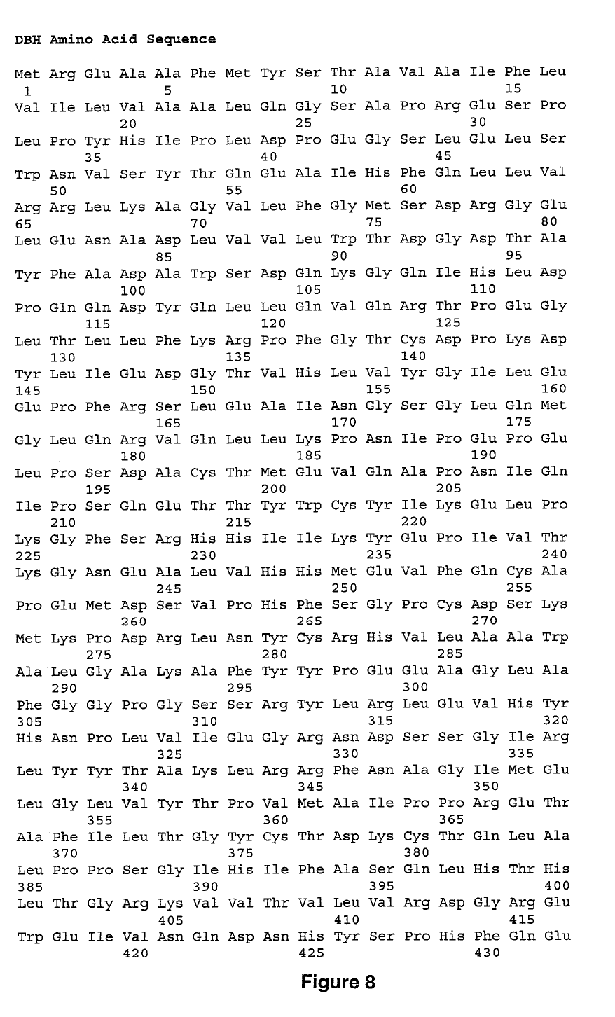

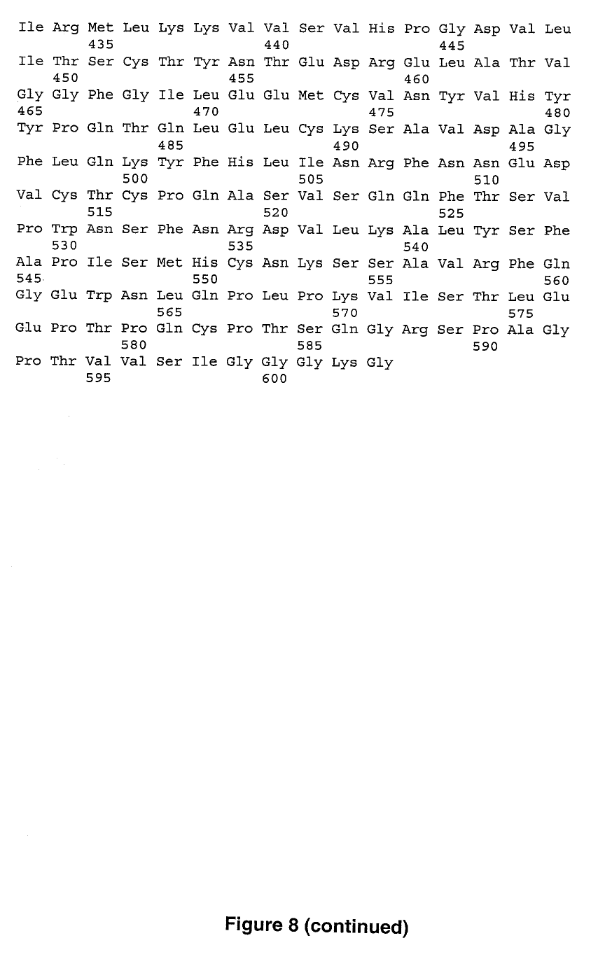

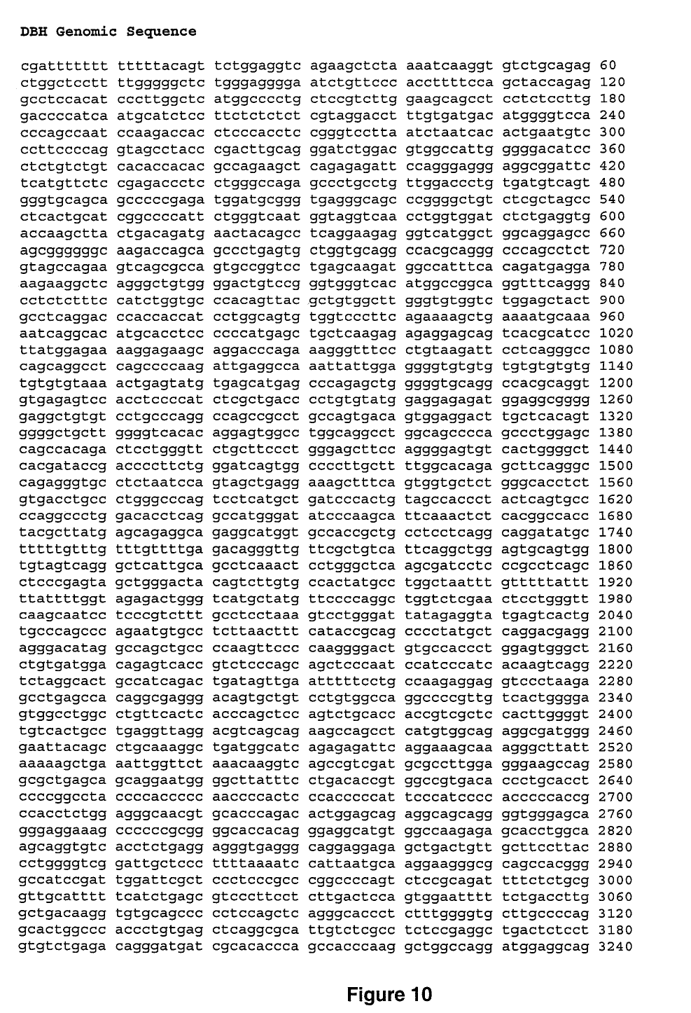

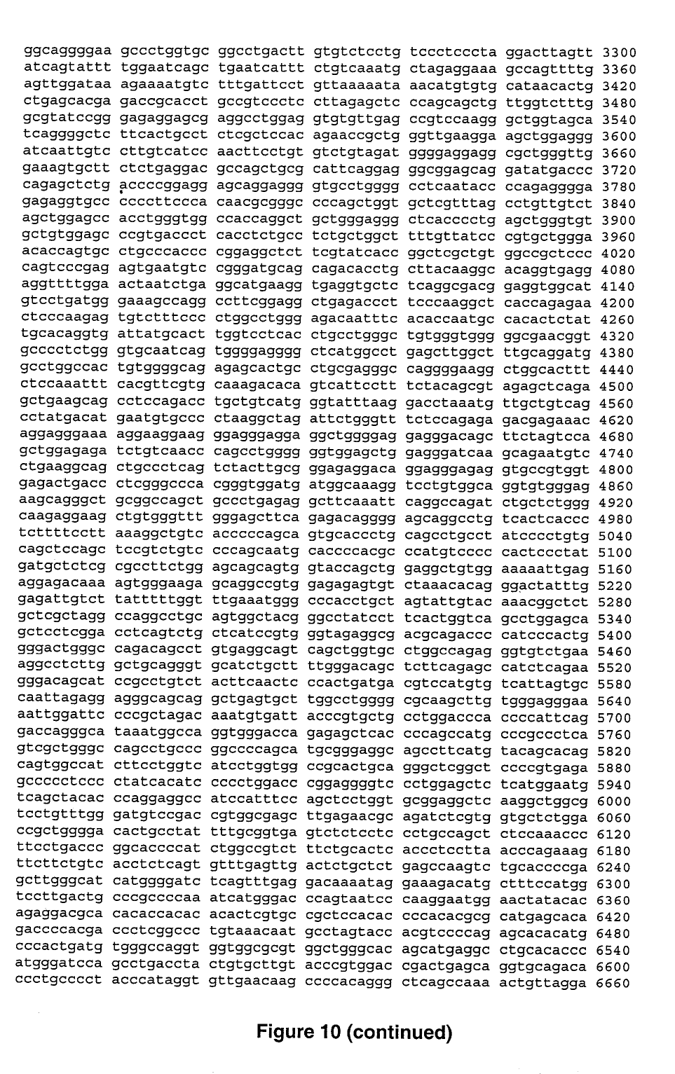

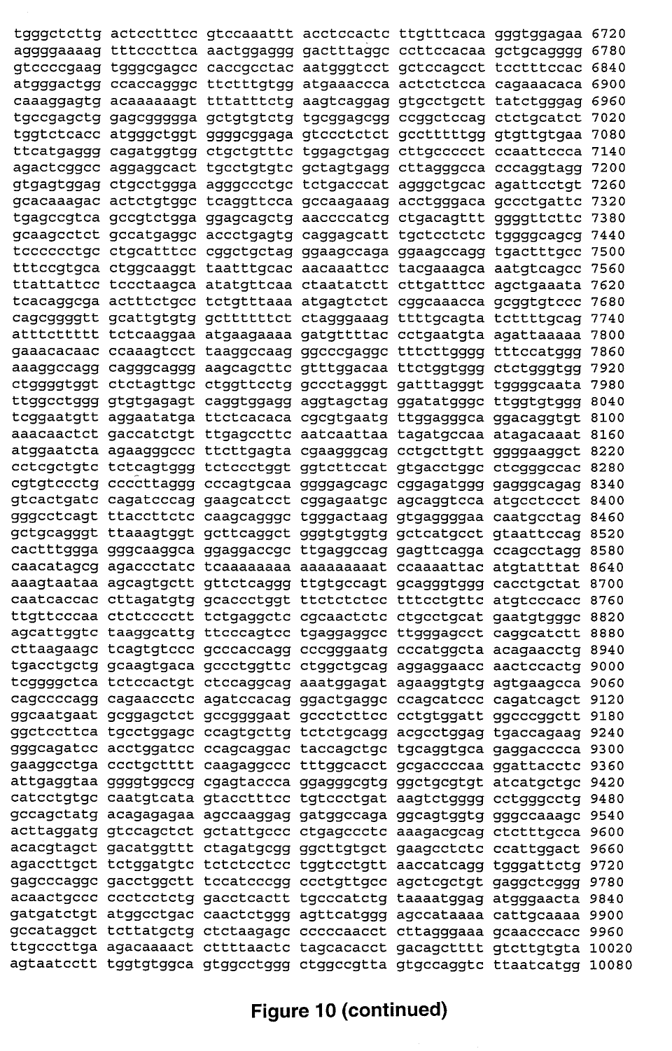

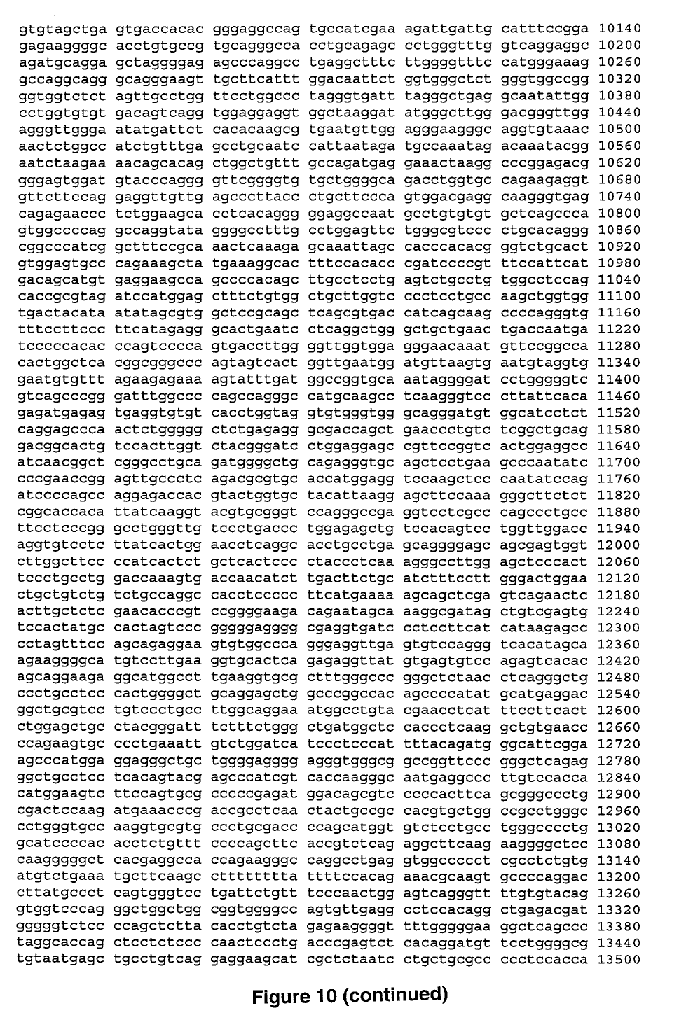

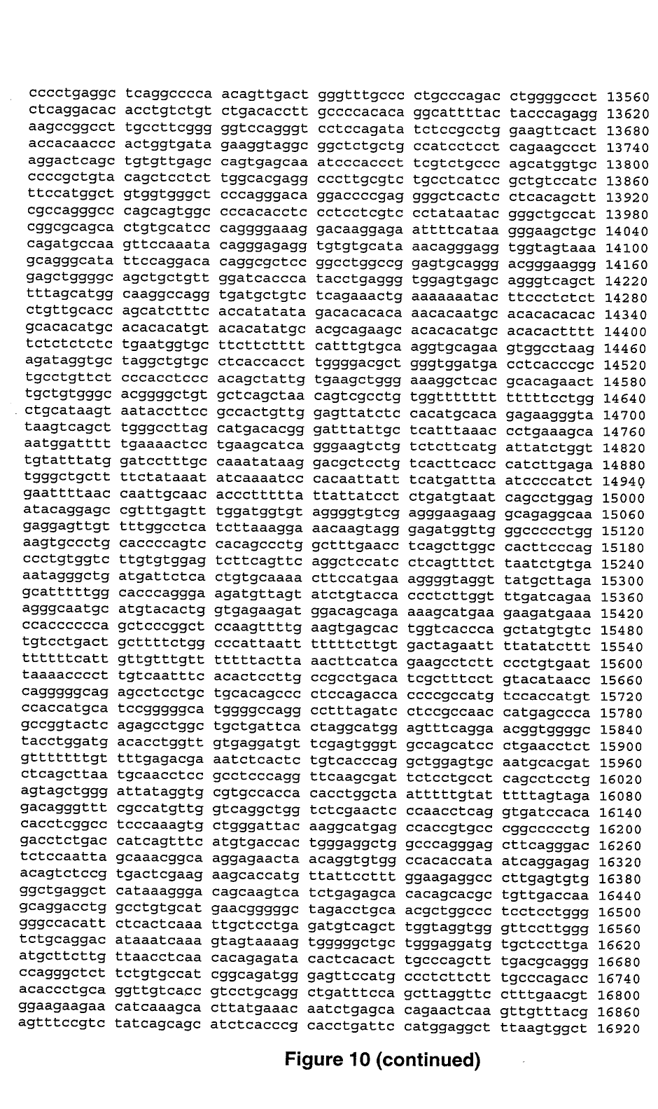

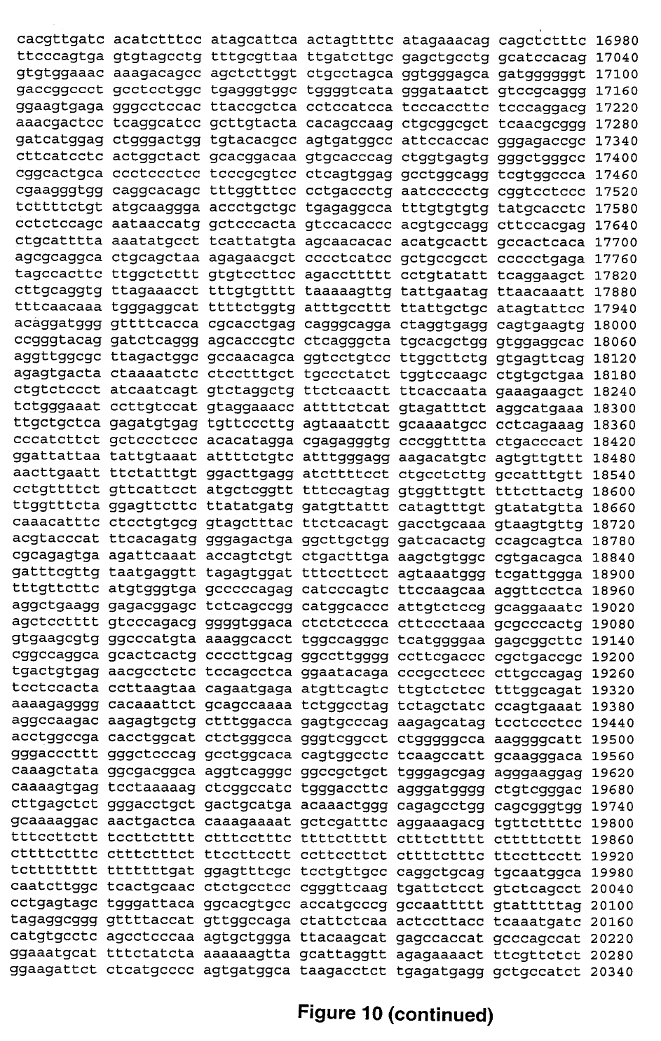

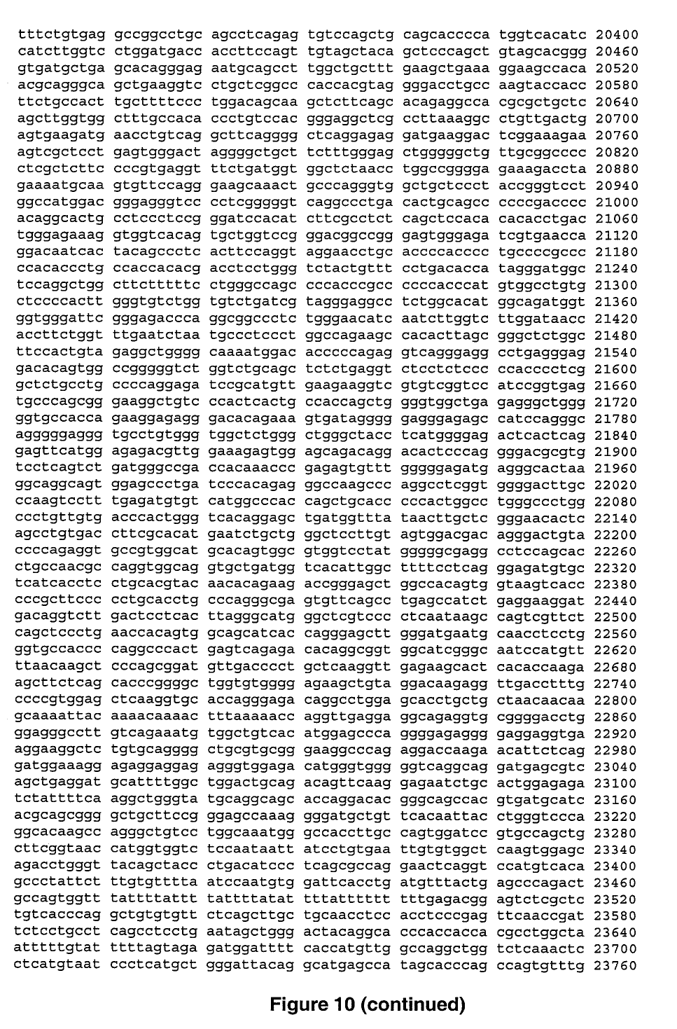

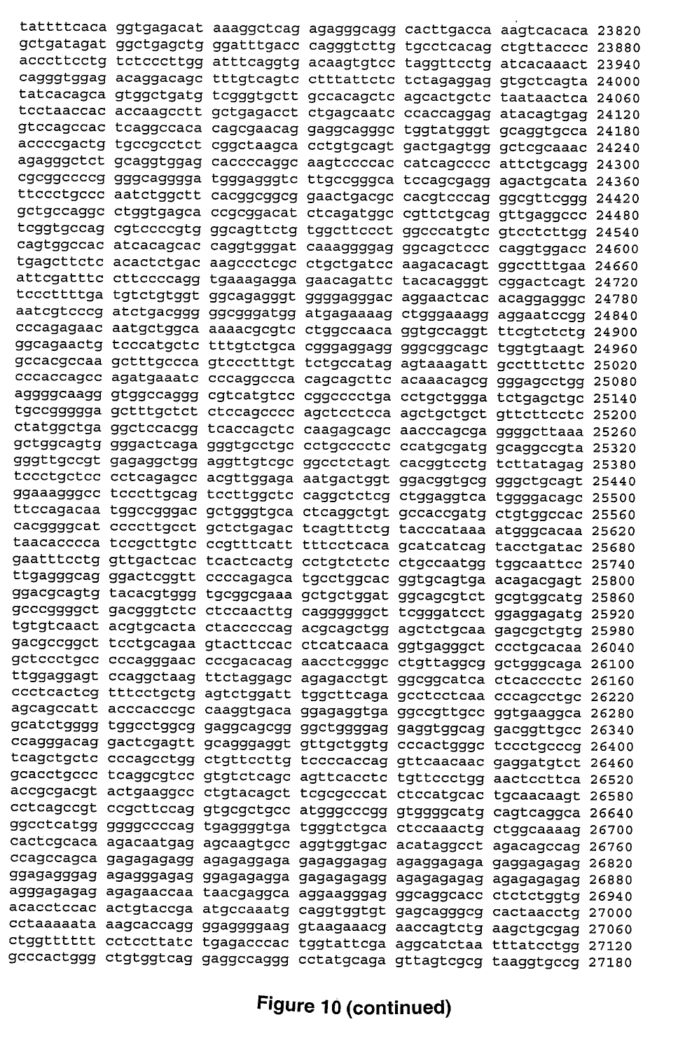

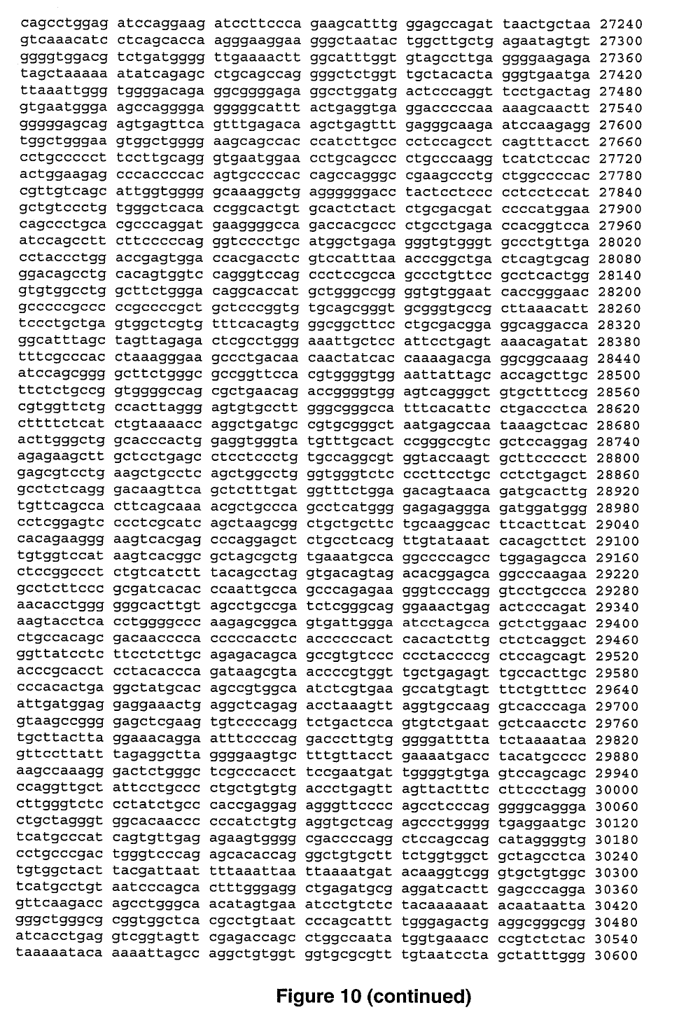

Norepinephrine (NE), a key neurotransmitter of the central and peripheral nervous systems, is synthesized by dopamine β-hydroxylase (DBH) (FIG. 8, SEQ ID NO:35), a biosynthetic enzyme encoded by the DBH polynucleotide sequence (FIG. 10, SEQ ID NO: 36). NE deficiency is a congenital disorder of unknown etiology, in which patients suffer profound autonomic failure (FIG. 7). Biochemical features of the syndrome include undetectable tissue and circulating levels of NE and epinephrine, elevated levels of dopamine, and undetectable levels of DBH.

We have identified seven novel variants including four potentially pathogenic mutations in human DBH from the analysis of two unrelated patients and their families. These patients are compound heterozygotes for variants affecting expression of DBH protein. Each patient carries one copy of a T to C transversion in the splice donor site of DBH intron 1, creating a premature stop codon. In patient 1, there is a missense mutation in DBH exon 2. Patient 2 carries missense mutations in exons 1 and 6 residing in cis. We have shown that in these patients NE deficiency is an autosomal recessive disorder that likely resulted from heterogeneous molecular lesions in DBH.

To explain the genetic basis of NE deficiency, we hypothesized that it might result from one or more mutations at the DBH locus, or at loci encoding critical regulators of DBH expression. The homeodomain transcription factors Phox2a and Phox2b belong to the latter group of candidate genes, because they are essential for development of noradrenergic neurons (Morin X, et al. Neuron; 18:411, 1997; Pattyn A, et al. Development; 124:4065, 1997) and for cell-specific transcription of DBH (Kim H S, et al. J Neurosci, 18:8247, 1998; Yang C, et al. J Neurochem, 71:1813, 1998). To address these possibilities, we designed oligonucleotide primers to amplify all of the exonic, peri-exonic, and proximal 5′ regions of the human DBH, Phox2a, and Phox2b genes in DNAs from two previously described cases of NE deficiency (Robertson D, et al. N Engl J Med, 314:1494, 1986; Biaggioni I, et al. Neurology, 40:370, 1990).

We identified seven novel variants including four potentially pathogenic polymorphisms in the DBH gene from two unrelated DBH deficiency patients. Based on genetic and functional analyses, we have demonstrated that NE deficiency is an autosomal recessive disorder resulting from heterogeneous molecular lesions in DBH. These results are important not only for elucidating a genetic origin of DBH deficiency, but also for indicating functionally relevant regions of the DBH protein. Such regions provide important therapeutic targets for rational drug design. In addition, the invention features methods for the identification of mutations residing in non-coding or coding regions of DBH. These mutations can indicate a noradrenergic disease, specifically DBH-deficiency, also depression, dementia, bipolar, schizophrenia, stillbirth, or attention deficit/hyperactivity disorder. Single mutations or combination of mutations identified herein can be used as genetic markers for noradrenergic-related disease such as congenital heart failure. These mutations can also be used for the development of drugs that modulate sympathetic nerve function, and for the development of drugs that modulate NE biosynthesis or inhibit DBH activity.

The following examples are for the purposes of illustrating the invention, and should not be construed as limiting, therefore. Below we describe the characterization of mutations in DBH, the biosynthetic enzyme that synthesizes NE from dopamine, and a target for compounds useful for the treatment of disorders that could benefit from a decrease in NE signaling, such as congestive heart failure.

Clinical and Neurochemical Features of NE-Deficient Patients

We collected DNA samples from two patients with NE deficiency, and sequenced all exons and exon/intron junctions of their DBH, Phox2a, and Phox2b genes. Resulting novel variants were then examined in the first degree relatives of the NE deficient patients, healthy European-American controls, and in patients with autonomic disorders other than NE deficiency. The first of the two patients was a 55-year old man with a lifelong history of fainting spells, ptosis, nasal stuffiness, and retrograde ejaculation. The second was a 48-year old woman with a lifelong history of orthostatic hypotension, ptsosis, nasal stuffiness, and a neonatal history of delayed eye opening. These patients have been previously reported (Robertson D, et al. N Engl J Med; 314:1494, 1986; Biaggioni I, et al. Neurology, 40:370, 1990). The patients were evaluated on the Vanderbilt General Clinical Research Center while in balance on a 150 mEq sodium and 80 mEq potassium diet one week after discontinuation of medications. Both direct and indirect hemodynamic monitoring was carried out and a variety of physiological and pharmacological tests were employed. Catecholamines and their metabolites were analyzed by radioenzymatic and liquid chromatographic methods as described previously (Robertson D, et al. N Engl J Med, 314:1494, 1986; O'Connor D T, et al. Clin Sci, 86:149, 1994).

Both patients experienced severe and reproducible orthostatic hypotension during the two weeks at the Vanderbilt General Clinical Research Center. Autonomic function tests revealed normal sinus arrhythmia, implying intact parasympathetic innervation of the heart. The expected blood pressure overshoot during phase IV of the Valsalva maneuver was absent, but the heart rate increased during the strain phase (phase II) of the Valsalva maneuver and this heart rate increase was blocked by atropine but not propranolol, indicating that its origin was in parasympathetic heart rate control. There existed a four-fold hypersensitivity to intravenous α₁-adrenoreceptor agonist (phenylephrine) and a three-fold hypersensitivity to the tachycardic effect of the β-adrenoreceptor agonist (isoproterenol). The α₂-adrenoreceptor agonist clonidine 0.3 mg po produced an increase in arterial pressure in these patients rather than the expected decrease. The α₂ antagonist, yohimbine, 10 mg po had no effect on blood pressure or plasma catecholamines. Sympathetic cholinergic function was intact as assessed by the thermoregulatory sweat test. There was an absent response to the indirectly acting sympathomimetic amine, tyramine (8 mg IV).

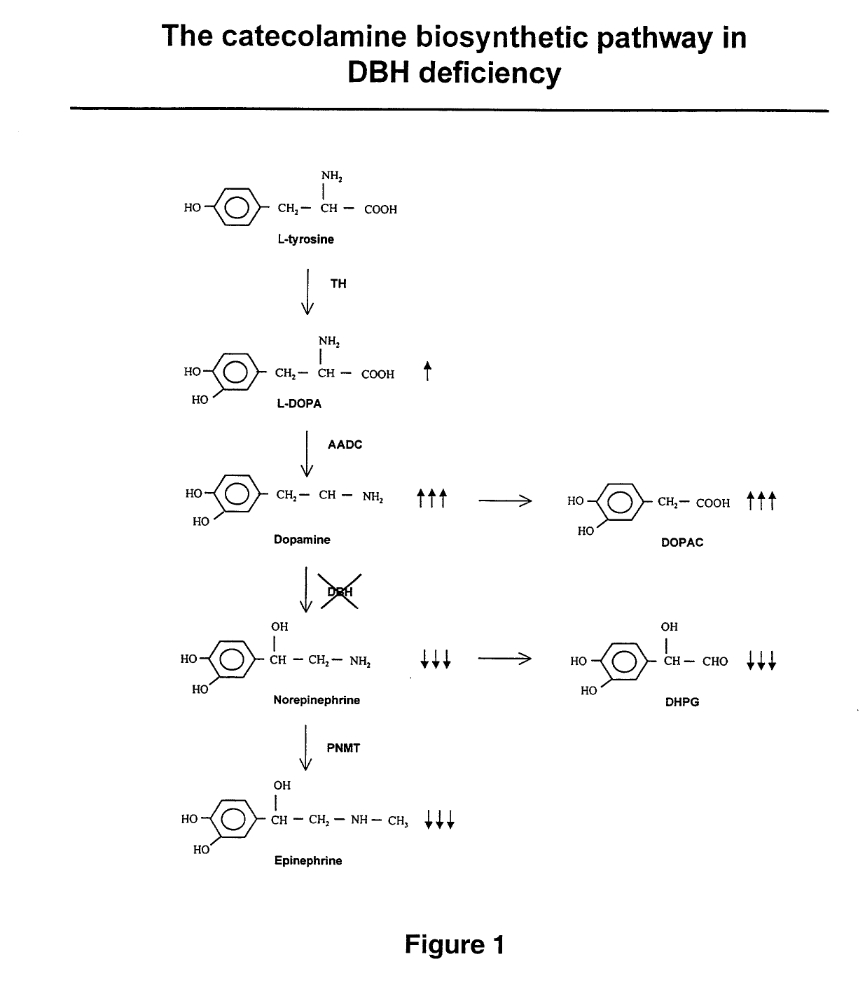

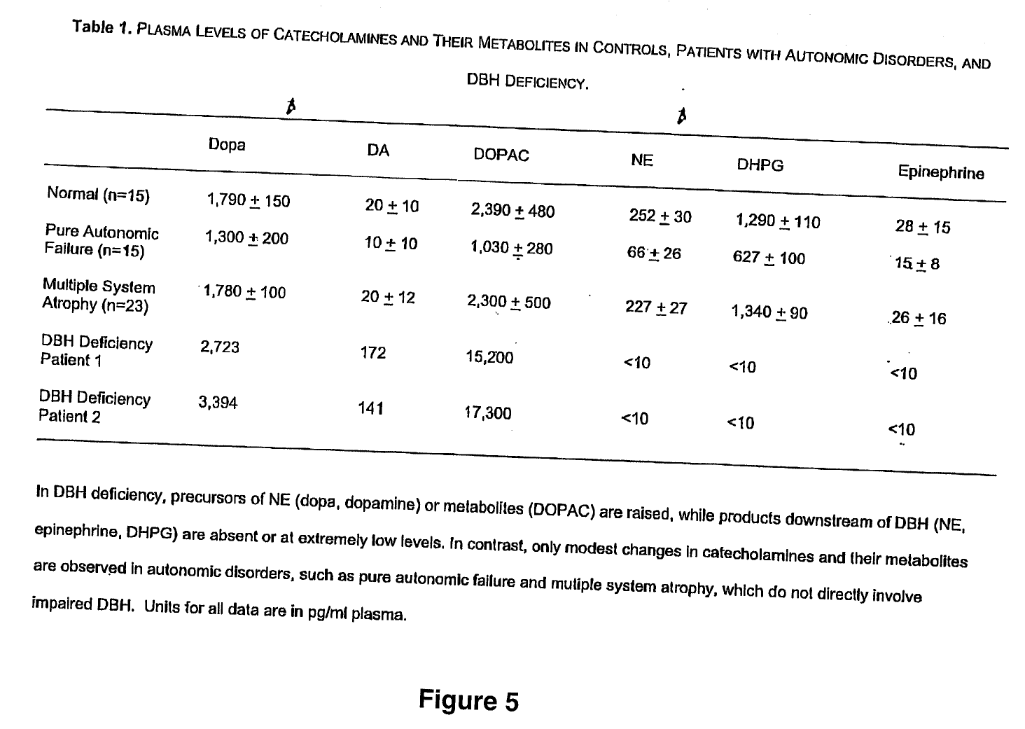

Plasma NE and its metabolite levels were strikingly abnormal. Plasma NE was below the limits of detection of the assay as was plasma epinephrine and plasma dihydroxyphenylglycol (DHPG). These findings indicate that the level of NE was less than 2% of normal and the level of DHPG was less than 1% of normal. In contrast, plasma dopamine was approximately ten-fold higher than normal and its precursor, dopa, was approximately two-fold higher. The dopamine metabolite, dihydroxyphenylacetic acid (DOPAC), was also four-fold elevated. Urinary catecholamine assay failed to detect NE or epinephrine. Analysis of plasma failed to detect DBH using either enzymatic or polyclonal antibody methods. Some of these findings are shown in FIG. 5. In sum, the neurochemical abnormalities observed in these patients indicate that the final step of NE biosynthesis is blocked (FIG. 1).

DBH Genetic Characterization

DNA samples were obtained from NE deficient patients and from 88 healthy individuals after informed consent was obtained. 6,443 bp of the DBH gene was PCR-amplified, including the proximal 1,468 bp of the 5′ upstream area, all 12 exons (2,744 bp), and 2,182 bp of intronic sequence spanning a minimum of 49 bp flanking each exon. The PCR reactions were performed as follows: initial denaturation occurred at 94° C. for 2 minutes, followed by 35 cycles of denaturation at 94° C. for 30 seconds, annealing at 55° C. for 30 seconds, and extension at 72° C. for 1 minute. The PCR products were then subjected to gel electrophoresis followed by polyacrylamide gel DNA extraction. Direct sequencing of the double-stranded PCR fragments was performed according to the thermal cycle sequencing protocol (Perkin Elmer). Detailed information on sequencing analysis of the DBH locus, including PCR primer sequences, are described in Zabetian et al., Am J Hum Genet, 68:515-522, 2001, hereby incoporated by reference.

Genotypes were determined by restriction fragment length polymorphisms (RFLPs). PCR products were digested with the appropriate restriction enzymes as follows: V87M—Nla III, IVS1+2T→C—Hph I, D100E—Bsa HI, IVS3+8C→T—Afl III, D331N—HinfI, and IVS10+415G→A—Sex AI. For the C-1021T polymorphism, we used the primer, 5′-AGCAGAATGTCCTGAAGGCAGCTGCCCCCAGTCTACTTG-3′(SEQ ID NO: 1), with a single nucleotide mismatch (underlined) to create an artificial Xcm I restriction site for genotyping. Digested PCR products were electrophoresed on 7% acrylamide gels, stained with ethidium bromide, imaged under UV transillumination, and photographed for genotype scoring.

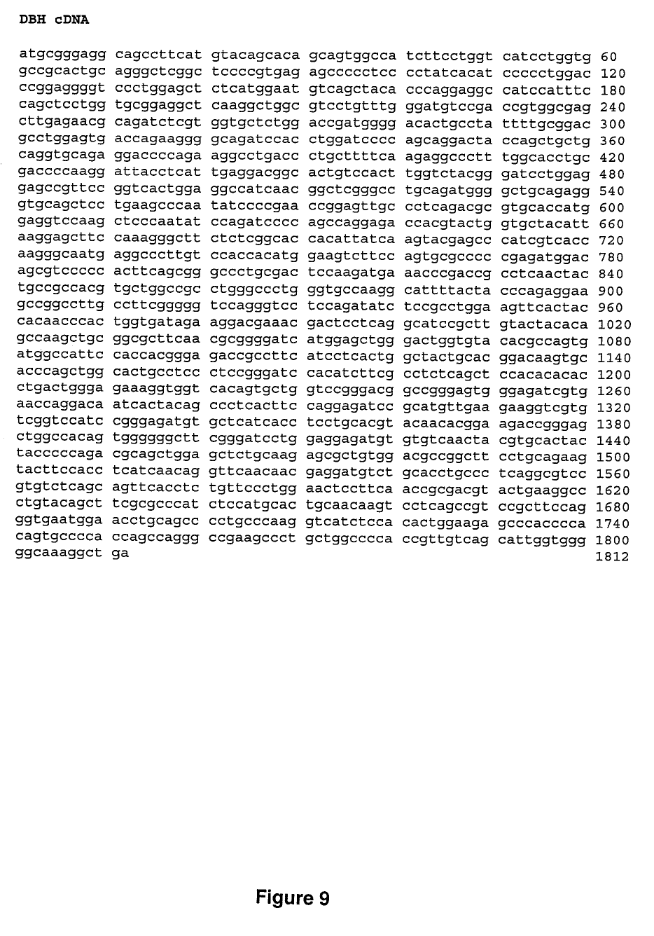

Human DBH cDNA (1.8 kb) (FIG. 9) was generated by RT-PCR from mRNA of human neuroblastoma SK-N-BE(2)C cells using a sense primer RDBH, 5′-TGCCCGAATTCGCCATGCGGGAGGCAGCCTTCATGTAC-3′ (SEQ ID NO: 2) and an antisense primer XDBA 5′ TAGGTCTCGAGTCAGCCTTTGCCCCCACCAATGCTG-3′ (SEQ ID NO: 3). The plasmid pcDNA-hDBH was constructed by digesting the cDNA with EcoRI and Xho I. This fragment was cloned into the mammalian expression vector pcDNA3.1/zeo (Invitrogen, Carlsbad, Calif.). The DBH genomic sequence (SEQ ID NO: 37) extending from exon 1 to intron 4 (7.2 kb) was amplified from both NE deficient patients and a healthy control using the primers RDBH and Ex4R, 5′-GACGGTGAAGCTGGGGAAAC-3′ (SEQ ID NO: 4). PCR products were completely digested with SfiI, and partially digested with EcoRI, and subcloned into the plasmid pcDNA-hDBH. The two resulting recombinant plasmids, pcDNA-DBH(E1/E4)-IVS1-wt and pcDNA-DBH(E1/E4)-IVS1-mut, were confirmed by sequencing. Human kidney HEK293 and monkey COS-7 cells were transfected using the calcium phosphate coprecipitation method as previously described (Kim K S, et al. J Neurosci, 14:7200, 1994). Poly(A)⁺ RNA was prepared from each cell line by oligo(dT)-cellulose affinity column chromatography (Kim C H, et al. J. Biol Chem, 274:6507, 1999), then reverse-transcribed with SUPERSCRIPT II RNase H-Reverse Transcriptase (Life Technologies, Inc) by priming with the oligonucleotide 3626DBA, 5′-CAATGAGGTAATCCTTGGGGTTCGCAGGTGCCAAAGG-3′ (SEQ ID NO: 5). An aliquot of the product was subjected to PCR using the primers 183DBH, 5′-CCTGGAGCTCTCATGGAATGTCAGCTACACCCAGG-3′(SEQ ID NO: 6) and 3626DBA. The mRNA splicing products from wild-type and mutant constructs were purified and directly cloned into the pGEM-T easy vector (Promega). Insert DNAs were isolated from individual colonies and sequenced.

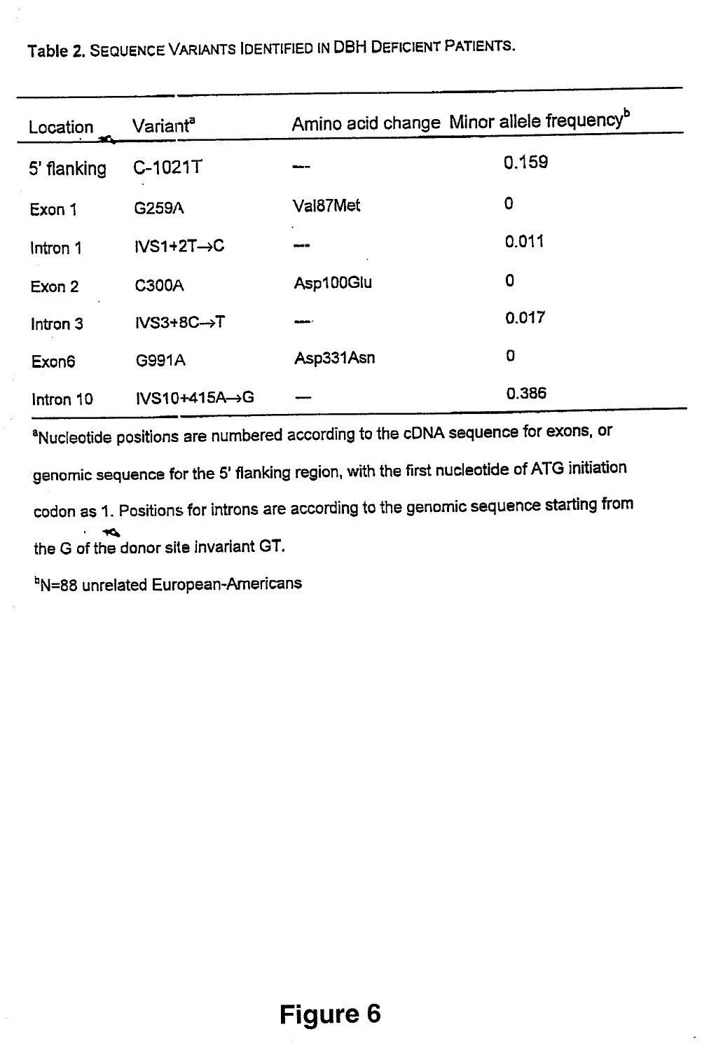

In DBH, we found seven novel single nucleotide polymorphisms (SNPs). Two of these SNPs, one located 1021 bp upstream of the ATG initiation codon and another in intron 10, were relatively common (FIG. 6). The remaining 5 SNPs in DBH appeared to be rare (FIG. 6), and were selected for further analysis. In contrast, we identified no mutations in Phox 2a or Phox 2b. Analyses of the candidate mutations at DBH, for each patient and first degree relatives, are described below.

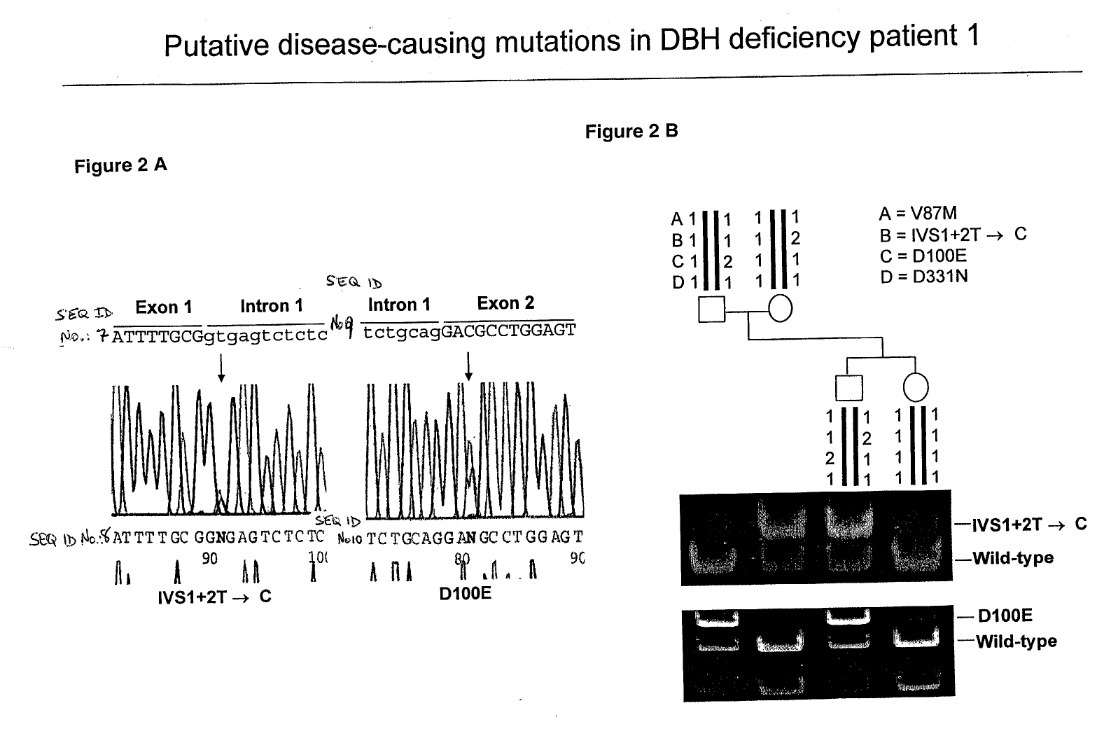

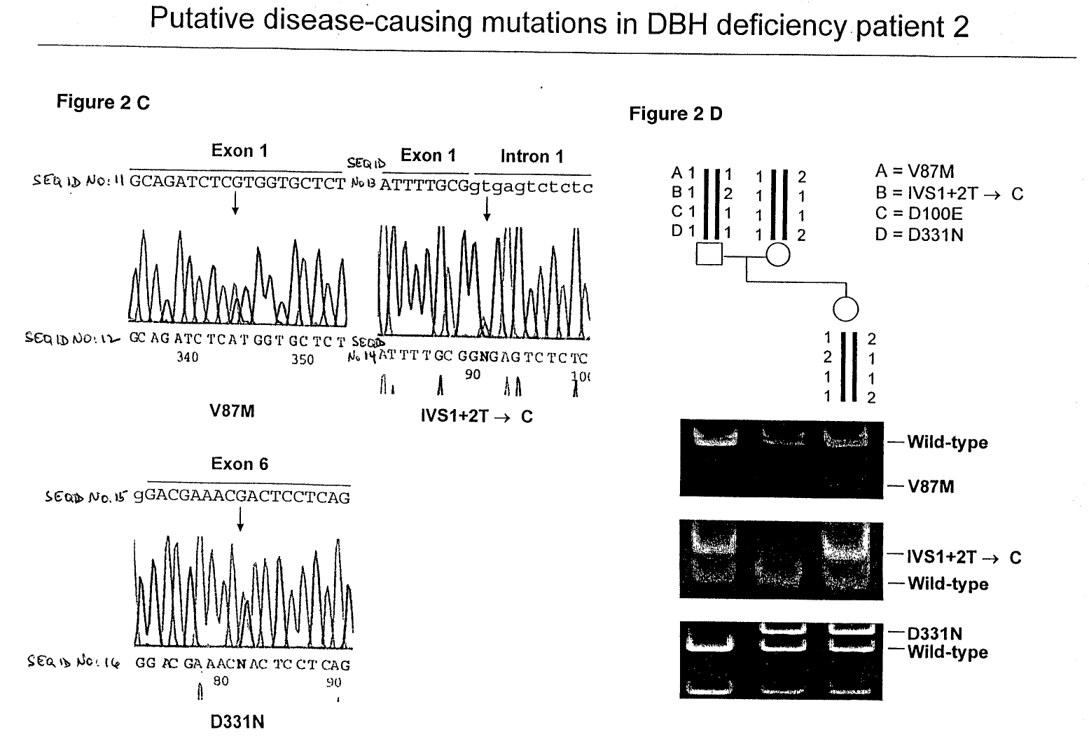

In Patient 1, single copies of four variants (C-1021 T, IVS1+2T→C, IVS3+8C→T, and IVS10+415G→A) were found in the non-coding region and one missense mutation (D100E) was found in exon 2. Among the mutations found in introns, IVS1+2T→C affects the invariant nucleotide T of the donor splice site GT dinucleotide, and is therefore expected to lead to aberrant splicing. IVS1+2T→C thus appears to contribute directly to the NE deficiency in Patient 1. We isolated plasmid clones containing IVS1+2T→C, and found that the single copies of both IVS3+8C→T and C-1021T reside in cis with IVS1+2T→C (data not shown). This observation was confirmed by pedigree analysis (FIG. 2). Thus, neither IVS3+8→T nor C-1021T appears necessary to explain lack of appropriate gene expression by the DBH haplotype on which they reside. Analysis of DBH genotypes in Patient 1 and first-degree relatives established that IVS10+415G→A also resides on the same chromosome as IVS1+2T→C (data not shown). These findings, that C-1021T and IVS10+415G→A are both common polymorphisms (FIG. 6), and that at least one unaffected parent of each patient is homozygous for each of these variants (data not shown), indicate that the identified polymorphisms cause NE deficiency. In contrast, the missense mutation in exon 2 (D100E) resides in trans to IVS1+2T→C, suggesting it might also contribute to NE deficiency in Patient 1. Consistent with this possibility, the E allele of D100E was not found in any of the control samples (FIG. 6). Furthermore, examination of familial genotypes showed that Patient 1 was the only first-degree relative in the pedigree that was a compound heterozygote for IVS1+2T→C and D100E (FIGS. 2A and B). Thus, in Patient 1, NE deficiency appeared to result from compound heterozygosity for the intronic splice-altering mutation, IVS1+2T→C, inherited from heterozygous mother, and the E allele of D100E, inherited from heterozygous father. Both parents were asymptomatic, strongly suggesting that NE deficiency is a recessive trait inherited at DBH by Patient 1.

Patient 2, like Patient 1, also carried a single copy of IVS1+2T→C (FIGS. 2C and D). Interestingly, the haplotype containing the shared mutation in the two patients was identical at C-1021T and at all three intronic mutations described above, suggesting that a single founder event gave rise to the mutation. Unlike Patient 1, Patient 2 carried the wild type D allele at both copies of D100E. However, sequencing revealed two novel missense mutations: V87M in exon 1 and D331N in exon 6. Plasmid cloning and pedigree analysis demonstrated that the mutations in V87M and D331N reside on the same chromosome, in trans to IVS1+2T→C (FIG. 2D).

Functional Analysis of IVS1+2T→C

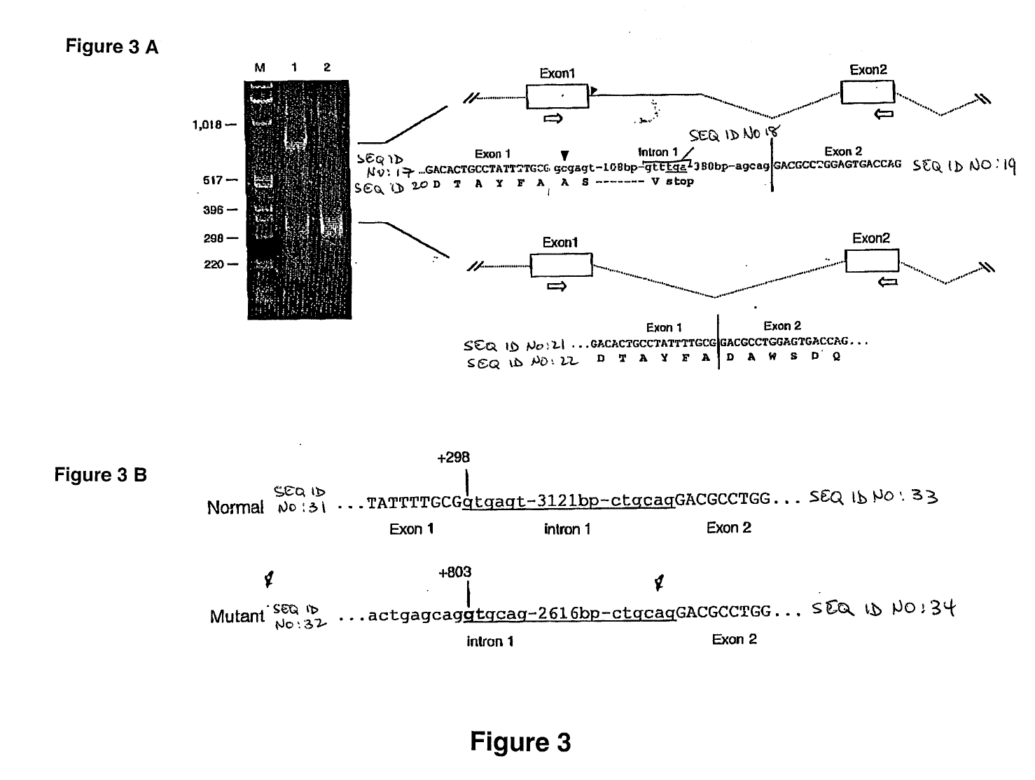

Given that both patients carry IVS1+2T→C at a consensus donor splice site (GT dinucleotide), we propose that this intronic mutation contributes to NE deficiency by disrupting appropriate splicing of DBH mRNA. To confirm this hypothesis, we subcloned a genomic fragment of the DBH gene encompassing exons 1 to 4 as well as all three corresponding introns, into the eukaryotic expression vector pcDNA3.1/zeo. We analyzed the mRNAs resulting from transient transfection of plasmids, containing the DBH gene fragment from either the normal or mutant allele, into COS-7 cells. As expected, RT-PCR analysis showed that the wild type allele construct generated a properly spliced RNA product of 311-bp length (FIG. 3A). The mutant construct, containing the IVS1+2T→C mutation, generated an 816-bp transcript that resulted from retention of 505 bp of intronic sequence, in addition to some normally spliced message (FIG. 3A). The identities of both the normal and abnormal transcripts were verified by sequencing. Thus, the mutation in the donor splice site at IVS1+2 led to the use of a cryptic donor splice site (GT) starting at IVS1+506. As a result, the aberrant transcript contains coding sequence for 39 abnormal amino acids followed by a premature stop codon (FIGS. 3A and B).

Cross-Species Sequence Comparisons

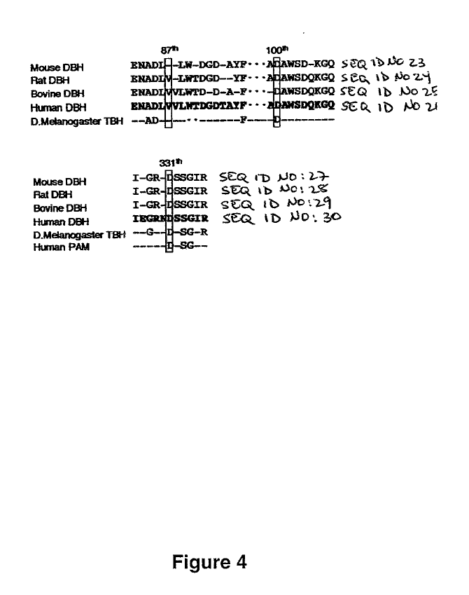

We compared published amino acid sequences among DBH proteins from different mammalian species to two functionally related proteins: D. melanogaster tyramine β-hydroxylase (TBH) and human peptidyl-glycine amidating monooxygenase (PAM) (FIG. 4) (Prigge S T, et al. Science, 278:1300, 1997; Monastirioti M, et al. J Neurosci, 16:3900, 1996). All of these enzymes require ascorbic acid and molecular oxygen for activity and belong to the copper-binding monooxygenase family. Aspartic acid at position 331, where a missense mutation was found in Patient 2, resides among two potential active sites of DBH that were previously identified by mechanism-based inhibitors (Fitzpatrick P F, et al. Arch Biochem Biophys, 257:231, 1987). This position was also in the middle of four putative copper-binding His-His and His-X-His motifs. Aspartic acid 331 is invariant among the six sequences compared. The existence of a genetic mutation at this amino acid and the conservation of this residue indicated the function importance of this amino acid. In addition, the aspartic acid at amino acid position 100, the site of the missense mutation in Patient 1, was also invariant among all five protein sequences. In contrast, valine at amino acid position 87 is variable, as murine DBH and D. melanogaster TBH contain different amino acids at this position.

NE deficiency was simultaneously recognized in the United States and the Netherlands in the mid-1980's (Robertson D, et al. N Engl J Med, 314:1494, 1986; Man in 't Veld A J, et al. Lancet, 1:183, 1987). Affected subjects displayed profound orthostatic hypotension. Subsequent studies suggested that the disorder resulted from a failure to express sufficient levels of DBH protein. In all NE deficient patients examined to date, NE and its metabolites, epinephrine and DHPG, were undetectable, while plasma levels of dopamine and its metabolites were significantly elevated (FIG. 5 and FIG. 1). DBH protein was undetectable in these patients by enzymatic assay (Robertson D, et al. N Engl J Med, 314:1494, 1986; Man in 't Veld A J, et al. Lancet, 1:183, 1987), radioimmunoassay (O'Connor, et al. Clin Sci, 86:149, 1994), or immunohistochemistry (Mathias C J, et al. Q J Med, 75:617, 1990). The autonomic and biochemical deficits of NE deficient patients could be successfully treated with the β-hydroxylated precursor of NE, dihydroxyphenylserine (DOPS) (Biaggioni J, et al. Lancet, 2:1170, 1987). The work presented herein provided genetic evidence that NE deficiency results from a failure to appropriately express DBH protein. This failure results from the presence of rare mutations at the DBH locus that likely alter DBH protein expression or structure.

A mutation at a consensus donor splice site, IVS1+2T→C, likely caused DBH deficiency in both patients. This variant lead to aberrant processing of mRNA by disrupting the consensus sequence at the splice-donor site of intron 1. However, at least in vitro, the mutation allowed some expression of properly spliced DBH message, consistent with previous studies showing that conversion of a splice-donor GT to GC can yield both normal and abnormal splice products (Aebi M, et al. Cell, 47:555, 1986; Mount S M, et al. Am J Hum Genet, 67:788, 2000). Thus, it is not entirely clear how IVS1+2T→C led to an apparently complete absence of DBH in NE deficiency. It is possible that in vivo processing of the aberrant sequence favored expression of the abnormal splice product to the exclusion of the normal splice product. Alternatively, it is possible that another variant at DBH, residing in cis to IVS1+2T→C, is necessary to produce the NE deficiency phenotype. Interestingly, single copies of three other variants residing on noncoding sequences (−1021C→T, IVS3+8C→T, and IVS10+415G→A) were found in the same haplotype containing IVS1+2T→C in both patients. Among these variants, the most likely candidate for a “second hit” mechanism is −1021C→T, because homozygosity at the T allele was strongly associated with very low plasma levels of DBH activity (Zabetian C P, et al. Am J Hum Genet, 68:515, 2001). This phenotype resulted from low circulating levels of DBH protein. These observations suggested the interesting possibility that NE deficiency arose, in part, through an effect requiring a specific haplotype, rather than a single variant.

Comparison of the DNA sequences of the putative disease-causing missense mutations to those of DBH from other species, as well as those of other copper-dependent mono-oxygenases, showed that both D100E (patient 1) and D331N (patient 2) occur at positions with no variations among all six amino acid sequences of DBH and related proteins (FIG. 4). In contrast, V87M (patient 2) occurred at a position with some variation among predicted amino acid sequences of related proteins. Therefore, D100E and D331N are the most likely candidates for pathogenic variants.

It is possible that the missense mutations identified in this study affect DBH protein activity or stability. Alternatively, it is possible that the identified missense mutations are pathogenic at the level of splicing, since they all reside in close proximity to the intron/exon junctions, and could, therefore, alter the efficiency or accuracy of splicing. Finally, the structural changes encoded by the putative disease-causing mutations could lead to improper intracellular processing of DBH protein. Altered post-translational processing could lead to a failure in the translocation of DBH into dense-core vesicles, the normal site of physiological DBH activity. Such a mechanism would result not only in failure of the vesicles to convert dopamine to NE, but could also account for the absence of DBH protein in plasma, CSF, and sympathetic fibers in NE-deficient patients.

Only a handful of cases of NE deficiency have been reported to date, suggesting that it is a rare disorder. However, the observation that frequent miscarriages and spontaneous abortions occurred in mothers of known NE deficient patients suggests that NE deficiency could be a clinically important cause of undiagnosed fetal and neonatal death (Man in 't Veld A J, et al. Lancet, 1:183, 1987; Robertson D, et al. Hypertension, 18:1, 1991). Neonatal hypotension, hypoglycemia, and hypothermia have all been observed in NE deficient subjects. Furthermore, targeted disruption of the DBH gene in mice produced homozygous (DBH^{-/-}) embryos that usually die in utero, only approximately 5% of such mice survived into adulthood (Thomas S A, et al. Nature, 374:643, 1995; Thomas S A, et al. J Neurochem, 70:2468, 1998). Treatment with DOPS, a therapy used in NE deficient patients, rescued homozygous mouse embryos, demonstrating that NE deficiency caused the observed mortality. DBH^{-/-} adult mice exhibited severe deficits in autonomic function, similar to those observed in human NE deficiency (Thomas S A, et al. J Neurochem, 70:2468, 1998). To address whether any of the identified DBH mutations occur at an appreciable frequency in the European-American population, we determined the DBH genotype of all candidate mutations in 88 unrelated healthy European-Americans. We identified two individuals who were heterozygous for the IVS1+2T→C variant. This frequency suggests that potentially fatal pre- and perinatal NE deficiency could be more common than has been appreciated based on the basis of its rarity in adults. Further study of the population genetics of NE deficiency-producing mutations, as well as direct surveys for such mutations at autopsy in unexplained spontaneous abortions, stillbirths, and newborn deaths, may elucidate whether NE deficiency is an epidemiologically significant cause of fetal and neonatal demise in humans.

In summary, we found seven novel variants including four potentially pathogenic mutations in the DBH gene from two unrelated NE deficient patients. IVS1+2T→C appeared to be a causative mutation in both patients. The second causative mutation at DBH apparently differed in the two patients; Patient 1 carried a missense mutation in exon 2, while Patient 2 had two missense mutations in exons 1 and 6. These observations indicate that NE deficiency is a Mendelian recessive disorder attributable to heterogeneous mutations at the DBH locus. These four mutations represent the first known examples of human variants that directly alter catecholamine biosynthesis.

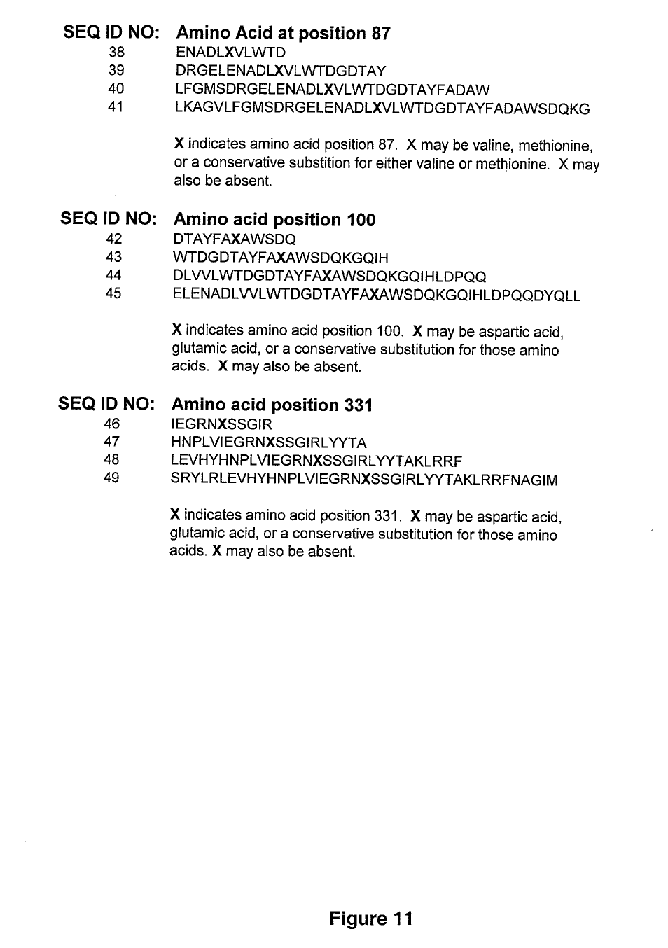

DBH mutations described herein are useful for identifying functionally important regions of the DBH polypeptide. Such regions include polypeptides shown in FIG. 11. These DBH polypeptide regions can be expressed and used to screen for compounds that bind DBH or inhibit DBH biological activity.

Polypeptide Expression

In general, polypeptides of the invention, e.g. regions of DBH, may be produced by transformation of a suitable host cell with all or part of a polypeptide-encoding nucleic acid molecule or region thereof in a suitable expression vehicle.

Those skilled in the field of molecular biology will understand that any of a wide variety of expression systems may be used to provide the recombinant protein. The precise host cell used is not critical to the invention. A polypeptide of the invention may be produced in a prokaryotic host (e.g., E. coli) or in a eukaryotic host (e.g., Saccharomyces cerevisiae, insect cells, e.g., Sf21 cells, or mammalian cells, e.g., NIH 3T3, HeLa, or preferably COS cells). Such cells are available from a wide range of sources (e.g., the American Type Culture Collection, Rockland, Md.; also, see, e.g., Ausubel et al., supra). The method of transformation or transfection and the choice of expression vehicle will depend on the host system selected. Transformation and transfection methods are described, e.g., in Ausubel et al. (supra); expression vehicles may be chosen from those provided, e.g., in Cloning Vectors: A Laboratory Manual (P. H. Pouwels et al., 1985, Supp. 1987).

One particular bacterial expression system for polypeptide production is the E. coli pET expression system (Novagen, Inc., Madison, Wis.). According to this expression system, DNA encoding a polypeptide is inserted into a pET vector in an orientation designed to allow expression. Since the gene encoding such a polypeptide is under the control of the T7 regulatory signals, expression of the polypeptide is achieved by inducing the expression of T7 RNA polymerase in the host cell. This is typically achieved using host strains that express T7 RNA polymerase in response to IPTG induction. Once produced, recombinant polypeptide is then isolated according to standard methods known in the art, for example, those described herein (Nagatsu T. Mol. Pharmacol, 16:529, 1979; Wimalasena, K et al., Anal. Biochem. 197:353, 1991).

Another bacterial expression system for polypeptide production is the pGEX expression system (Pharmacia). This system employs a GST gene fusion system which is designed for high-level expression of genes or gene fragments as fusion proteins with rapid purification and recovery of functional gene products. The protein of interest is fused to the carboxyl terminus of the glutathione S-transferase protein from Schistosoma japonicum and is readily purified from bacterial lysates by affinity chromatography using Glutathione Sepharose 4B. Fusion proteins can be recovered under mild conditions by elution with glutathione. Cleavage of the glutathione S-transferase domain from the fusion protein is facilitated by the presence of recognition sites for site-specific proteases upstream of this domain. For example, proteins expressed in pGEX-2T plasmids may be cleaved with thrombin; those expressed in pGEX-3X may be cleaved with factor Xa.

Once the recombinant polypeptide of the invention is expressed, it is isolated, e.g., using affinity chromatography. In one example, an antibody (e.g., produced as described herein) raised against a polypeptide of the invention may be attached to a column and used to isolate the recombinant polypeptide. Lysis and fractionation of polypeptide-harboring cells prior to affinity chromatography may be performed by standard methods (see, e.g., Ausubel et al., supra).

Once isolated, the recombinant protein can, if desired, be further purified, e.g., by high performance liquid chromatography (see, e.g., Fisher, Laboratory Techniques In Biochemistry And Molecular Biology, eds., Work and Burdon, Elsevier, 1980).

Polypeptides of the invention, particularly short peptide fragments, can also be produced by chemical synthesis (e.g., by the methods described in Solid Phase Peptide Synthesis, 2nd ed., 1984 The Pierce Chemical Co., Rockford, Ill.). Also included in the invention are polypeptides which are modified in ways which do not abolish their pathogenic activity (assayed, for example as described herein). Such changes may include certain mutations, deletions, insertions, or post-translational modifications, or may involve the inclusion of any of the polypeptides of the invention as one component of a larger fusion protein.

The invention further includes analogs of any naturally-occurring DBH polypeptide of the invention. Analogs can differ from the naturally-occurring DBH polypeptide by amino acid sequence differences, by post-translational modifications, or by both. Analogs of the invention will generally exhibit at least 85%, more preferably 90%, and most preferably 95% or even 99% identity with all or part of a naturally-occurring amino acid sequence of the invention. The length of sequence comparison is at least 15 amino acid residues, preferably at least 25 amino acid residues, and more preferably more than 35 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., β or γ amino acids.

Methods useful for producing full-length polypeptides, may also be used to produce a fragment of the DBH polypeptides of the invention. As used herein, the term “fragment,” means at least 5, preferably at least 20 contiguous amino acids, preferably at least 30 contiguous amino acids, more preferably at least 50 contiguous amino acids, and most preferably at least 60 to 80 or more contiguous amino acids. DBH fragments can be generated by methods known to those skilled in the art or may result from normal protein processing (e.g., removal of amino acids from the nascent polypeptide that are not required for biological activity or removal of amino acids by alternative mRNA splicing or alternative protein processing events). The aforementioned general techniques of polypeptide expression and purification can also be used to produce and isolate useful peptide fragments or analogs (described herein).

DBH Polypeptide Regions

Mutations described herein identify functionally important regions of the DBH polypeptide. Such regions include the polypeptides shown in FIG. 11. The minimum number of consecutive amino acids that forms a region is desirably at least 10 amino acids. A DBH polypeptide region can include amino acids 87-515 of SEQ ID NO:35 and the putative catalytic core domain (Oyarce, J. Biol. Chem. 276:33265, incorporated herein by reference).

Screening Assays

Candidate compounds may be screened for those that specifically bind to and inhibit DBH. The efficacy of such a candidate compound is dependent upon its ability to interact with a region of the DBH polypeptide. Such an interaction can be readily assayed using any number of standard binding techniques and functional assays (e.g., those described in Ausubel et al., supra). For example, a candidate compound may be tested in vitro for interaction and binding with a polypeptide of the invention and its ability to modulate DBH may be assayed by any standard assays (e.g., those described herein, for example, Nagatsu T. Mol. Pharmacol, 16:529, 1979 and Wimalasena, K et al., Anal. Biochem. 197:353, 1991).

Methods for detecting binding of a candidate compound to DBH will be known to those of skill in the art. Binding may be detected either directly or indirectly. If desirable, various labels can be used as means for detecting binding of DBH to a candidate compound. DBH can be directly or indirectly detectably labeled, for example, with a radioisotope, a fluorescent compound, a bioluminescent compound, a chemiluminescent compound, a metal chelator or an enzyme. Those of ordinary skill in the art will know of other suitable labels or will be able to ascertain such, using routine experimentation.

The DBH/candidate compound complex may be purified using methods known to those of skill in the art. Such methods may include contacting DBH with a suitable antibody, a size selection, or chromatographic separation. The candidate compound can then be separated from the purified complex and identified using standard methods known to one of skill in the art.

In one example, a candidate compound that binds to a DBH polypeptide region may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for a region of the DBH polypeptide is identified on the basis of its ability to bind to a region of the polypeptide and be immobilized on the column. To isolate the compound, the column is washed to remove non-specifically bound molecules, and the compound of interest is then released from the column and collected. Compounds isolated by this method (or any other appropriate method) may, if desired, be further purified (e.g., by high performance liquid chromatography). In addition, these candidate compounds may be tested for their ability to inhibit DBH biological activity using a standard enzyme assay, for example, Nagatsu T. Mol Pharmacol, 16:529, 1979 and Wimalasena, K et al., Anal Biochem 197:353, 1991. Compounds isolated by this approach may also be used, for example, as therapeutics to treat disease. Compounds that bind to a DBH polypeptides with an affinity constant less than or equal to 10 mM are considered particularly useful in the invention.

Test Compounds and Extracts

In general, compounds capable of inhibiting enzyme activity are identified from large libraries of both natural product or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to have a DBH inhibitory, or a binding activity, further fractionation of the positive lead extract is necessary to isolate chemical constituents responsible for the observed effect. Thus, the goal of the extraction, fractionation, and purification process is the careful characterization and identification of a chemical entity within the crude extract having anti-pathogenic activity. Methods of fractionation and purification of such heterogenous extracts are known in the art. If desired, compounds shown to be useful for inhibiting DBH biological activity may be chemically modified according to methods known in the art.

Pharmaceutical Therapeutics

The invention provides a simple means for identifying compounds (including peptides, small molecule inhibitors, and mimetics) capable of inhibiting the activity of DBH. Accordingly, a chemical entity discovered to have medicinal value using the methods described herein are useful as either drugs or as information for structural modification of existing DBH inhibitory compounds, e.g., by rational drug design.

For therapeutic uses, the compositions or agents identified using the methods disclosed herein may be administered systemically, for example, formulated in a pharmaceutically-acceptable buffer such as physiological saline. Treatment may be accomplished directly, e.g., by treating the animal with antagonists that disrupt, suppress, attenuate, or neutralize the DBH polypeptide. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of an anti-pathogenic agent in a physiologically-acceptable carrier. Suitable carriers and their formulation are described, for example, in Remington's Pharmaceutical Sciences by E. W. Martin. The amount of the anti-pathogenic agent to be administered varies depending upon the manner of administration, the age and body weight of the patient, and with the type of disease and extensiveness of the disease. Generally, amounts will be in the range of those used for other agents used in the treatment of other microbial diseases, although in certain instances lower amounts will be needed because of the increased specificity of the compound. A compound is administered at a dosage that inhibits DBH enzyme activity. For example, for systemic administration a compound is administered typically in the range of 0.1 ng-10 g/kg body weight.

All publications mentioned in this specification are herein incorporated by reference to the same extent as if each independent publication was specifically and individually indicated to be incorporated by reference.

Other Embodiments

While the invention has been described in connection with specific embodiments thereof, it will be understood that it is capable of further modifications. This application is intended to cover any variations, uses, or adaptations following, in general, the principles of the invention and including such departures from the present disclosure within known or customary practice within the art to which the invention pertains and may be applied to the essential features hereinbefore set forth.

What is claimed is:

1. A method for determining whether a compound is a potentially useful dopamine β-hydroxylase inhibitor by
a. contacting said compound with a dopamine β-hydroxylase polypeptide region that comprises an amino acid corresponding to amino acid position 87, amino acid position 100, or amino acid position 331 of SEQ ID NO:35; and
b. detecting binding of said compound to said region, wherein binding indicates that said compound is potentially an inhibitor of dopamine β-hydroxylase.

2. The method of claim 1, wherein said amino acid corresponding to amino acid position 87 is methionine, valine, isoleucine, or leucine.

3. The method of claim 1, wherein said amino acid corresponding to amino acid position 100 is glutamic acid, aspartic acid, asparagine, or glutamine.

4. The method of claim 1, wherein said amino acid corresponding to amino acid position 331 is aspartic acid, glutamic acid, asparagine, or glutamine.

5. The method of claim 1, wherein said region comprises 5-20 amino acids on either side of an amino acid corresponding to amino acid position 87, 100, or 331 of SEQ ID NO: 35.

6. A method for determing whether a compound is a potentially useful dopamine β-hydroxylase inhibitor by
a. contacting said compound with a dopamine β-hydroxylase polypeptide region that comprises an amino acid corresponding to amino acid position 87, amino acid position 100, or amino acid position 331 of SEQ ID NO:35; and
b. detecting dopamine β-hydroxylase biological activity, wherein binding indicates that said compound is potentially an inhibitor of dopamine β-hydroxylase.

7. The method of claim 6, wherein said amino acid corresponding to amino acid position 87 is methionine, valine, isoleucine, or leucine.

8. The method of claim 6, wherein said amino acid corresponding to amino acid position 100 is glutamic acid, aspartic acid, asparagine, or glutamine.

9. The method of claim 6, wherein said amino acid corresponding to amino acid position 331 is aspartic acid, glutamic acid, asparagine, or glutamine.

10. The method of claim 6, wherein said region comprises 5-20 amino acids on either side of an amino acid corresponding to amino acid position 87, 100, or 331.

11. The method of claim 6, wherein said DBH biological activity is norepinephrine biosynthesis.

12. The method of claim 1, wherein said candidate compound is useful for the treatment of a patient with congestive heart failure, or chronic activation of sympathetic nerve function.

13. The method of claim 6, wherein said candidate compound is useful for the treatment of a patient with congestive heart failure, or chronic activation of sympathetic nerve function.

14. The method of claim 1, wherein said inhibitor increases dopamine levels.

15. The method of claim 6, wherein said inhibitor increases dopamine levels.

16. The method of claim 14, wherein said increase in dopamine levels benefits renal function in a patient with congestive heart failure.

17. The method of claim 15, wherein said increase in dopamine levels benefits renal function in a patient with congestive heart failure.

18. An isolated polypeptide region comprising the sequence of SEQ ID NO:38, 42, or 46.

19. A method for determining whether a patient has an increased risk of miscarriage, still birth, or fetal or neonatal death, said method comprising determining whether a dopamine β-hydroxylase polynucleotide sequence of said patient has a mutation at the consensus donor site between the first exon and first intron, or in a polynucleotide that encodes a region comprising either aspartic acid at amino acid position 100, aspartic acid at amino acid position 331, or valine at amino acid position 87, wherein a mutation indicates that said patient has an increased risk for having a miscarriage, still birth, or fetal or neonatal death.

20. A method for determining whether a patient has an increased risk of noradrenergic disease, depression, dementia, bipolar disorder, schizophrenia, or attention deficit/hyperactivity disorder, said method comprising determining whether a dopamine β-hydroxylase polynucleotide sequence of said patient has a mutation at the consensus donor site between the first exon and first intron, or in a polynucleotide that encodes a fragment comprising either aspartic acid at amino acid position 100, aspartic acid at amino acid position 331, or valine at amino acid position 87, wherein a mutation indicates that said patient has an increased risk for having noradrenergic disease, depression, dementia, bipolar disorder, schizophrenia, or attention deficit/hyperactivity disorder.
